(19) United States
(12) Patent Application Publication
(10) Pub. No.: US 2019/0351198 A1
(43) Pub. Date: Nov. 21, 2019

(54) CATHETERS WITH STRUCTURALLY SUPPORTED EXPANDABLE ELEMENTS AND METHODS FOR SAME

(71) Applicant: Surmodics, Inc., Eden Prairie, MN (US)

(72) Inventors: Conleth A. Mullen, Galway (IE); Michael J. Finnerty, Oranmore (IE); Colm C. Manning, Ballinasloe (IE); Noel M. Gately, Roscommon (IE); Noel C. Fox, Athenry (IE); Nathan Lockwood, Minneapolis, MN (US); Joram Slager, St. Louis Park, MN (US); Amy Trocke, Plymouth, MN (US); Karl V. Ganske, Hopkins, MN (US)

(21) Appl. No.: 16/414,597

(22) Filed: May 16, 2019

Related U.S. Application Data
(60) Provisional application No. 62/672,512, filed on May 16, 2018.

(51) Int. Cl. A61M 25/10; A61F 2/24; A61M 29/02

(52) U.S. Cl. CPC A61M 25/1002; A61F 2/2433; A61M 29/02; A61M 2025/1086; A61M 2025/105; A61M 2025/1075; A61M 2025/1093; A61M 2205/0216

(57) ABSTRACT

A balloon catheter includes a catheter shaft extending between proximal and distal end portions along a shaft axis. The catheter shaft includes an inflation lumen, and at least one inflation port in communication with the inflation lumen. A balloon assembly is coupled with the catheter shaft and in communication with the at least one inflation port. The balloon assembly includes a balloon membrane having a balloon body, a balloon proximal nose and a balloon distal nose coupled with the catheter shaft. An interlaced jacket is coupled with the balloon membrane, the interlaced jacket includes interlaced filaments extending at diverging angles relative to the shaft axis.

### CLAIM OF PRIORITY

This application claims the benefit of priority to U.S. Provisional Application Ser. No. 62/672,512, filed on May 16, 2018, which is incorporated herein by reference in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright Surmodics, Inc.; Eden Prairie, Minn. All Rights Reserved.

### TECHNICAL FIELD

This document pertains generally, but not by way of limitation, to catheters with structurally supported expandable elements, such as balloons used with balloon catheters.

### BACKGROUND

Balloon catheters, such as percutaneous transluminal angioplasty (PTA) balloon catheters, are constructed with robust materials in the balloon to provide semi-compliant and non-compliant inflation (e.g., in contrast to elastomeric behavior with compliant balloons when inflated). The balloons are inflated within cavities and passages (including vessels) to dilate these spaces and accordingly provide therapeutic benefits, such as increased blood flow.

In some examples, balloons include fibers extending in one or more of longitudinal (e.g., along the balloon length) or circumferential directions (e.g., along the perimeter of the balloon) to constrain inflation of the balloon to a specified shape while subject to high pressures (e.g., 20 atmospheres or more). The fibers impart structural rigidity to the balloon and facilitate the inflation of the balloon to high pressures while limiting failure.

### OVERVIEW

The present inventors have recognized, among other things, a problem with high pressure balloon catheters. Some example catheters have balloons that include fibers oriented in one or more directions, for instance along a longitudinal axis. The fibers enhance the strength of the balloons and allow for operation at high pressure. The fibers constrain inflation to a specified shape and decrease failure of the balloon (e.g., radial blow outs). Additionally, the fibers increase the stiffness (e.g., rigidity) of the balloon. The increased stiffness of the balloon in combination with inflation to high pressures, such as 20 atmospheres or more, causes patient discomfort and, in some examples, acute pain. However, inflation of the balloon to high pressures facilitates the desired therapeutic outcome (e.g., dilation of a vessel) and accordingly the patient may be subjected to one or more of discomfort or pain in the interest of realizing the desired outcome.

The present subject matter can help provide a solution to this problem, such as by providing a balloon catheter (e.g., having an inflatable balloon membrane, multiple cellular balloon, reticulated substrate or the like) having an assembly that includes one or more of a balloon, a compressible (or expandable) substrate or the like having structural support configured to increase the strength of the assembly (e.g., permit inflation pressures of 20 atmospheres or more, 30 atmospheres or more, 40 atmospheres or more or the like) and at the same time retain flexibility to permit use within tortuous vasculature in a more comfortable manner than other balloon catheters.

In one example, a balloon catheter has a balloon membrane and an interlaced jacket provided along at least a portion of the balloon membrane. The interlaced jacket includes interlaced filaments extending at diverging angles relative to a catheter shaft. For instance, the interlaced filaments are not aligned with a longitudinal axis of the catheter (e.g., the catheter shaft or the balloon membrane along the catheter shaft). In another example, diverging angles include filaments oriented in a manner including one or more of filaments diverging from or converging toward the longitudinal axis of the catheter (and accordingly are not aligned with the longitudinal axis).

The interlaced jacket enhances the strength of the balloon membrane (allowing inflation at pressures of 20 atmospheres or more) while minimizing failure of the balloon membrane. Further, the interlaced jacket provides a constraint to inflation of the balloon membrane that guides inflation to a specified balloon profile (e.g., size, shape, shape or taper of one or more of the balloon noses or the like). Furthermore, while constraining inflation of the balloon membranes (at pressures of 20 atmospheres or more) the interlacing of the jacket at angles relative to the longitudinal axis maintains flexibility in the balloon catheter, assists in traversing tortuous vasculature while inflated and accordingly enhances patient comfort.

Additionally, the interlaced jacket is used in some examples with compliant balloon membranes (e.g., configured to stretch when inflated) to evenly distribute stresses in the balloon membranes through deformation of the membrane because of its compliance. Because of the constraint provided by the interlaced jacket, the compliant balloon membrane reliably inflates and distributes stresses across the membrane while minimizing the thinning of the compliant (e.g., stretchable) membrane that may otherwise cause rupture.

In another example, a catheter includes an assembly constructed with a compressible substrate (e.g., a foam elastomer) having reticulated pores. The compressible substrate is formed into a specified profile (e.g., shape, size or the like) with the reticulated pores in a neutral configuration, filled with a fluid, such as nitrogen, saline or the like. A vacuum source (pump or the like) draws the fluid from the reticulated pores and accordingly transitions the compressible substrate from the deployed (e.g., expanded) configuration to a compressed configuration. The compressible substrate is readily formed into a specified profile that maintains its shape and size while retaining flexibility because of compressible nature of the substrate.

This overview is intended to provide an overview of subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view showing one example of a catheter assembly including at least one deployable element.

FIG. 2 is a schematic view of the catheter assembly of FIG. 1 in a deployed configuration within vasculature.

FIG. 3A is a schematic view of a balloon of a catheter assembly including a jacket having longitudinally extending filaments.

FIG. 3B is a schematic view of the deployable element of the catheter assembly of FIG. 1 including filaments extending at diverging angles.

FIG. 4A is a schematic view of a balloon assembly having an interlaced jacket including braided filaments.

FIG. 4B is a schematic view of a balloon assembly having an interlaced jacket including woven filaments.

FIG. 4C is a schematic view of a balloon assembly having an interlaced jacket including knit filaments.

FIG. 4D is a schematic view of a balloon assembly having an interlaced jacket including one example of crocheted filaments.

FIG. 4E is a schematic view of a balloon assembly having an interlaced jacket including one example of nalbound filaments.

FIG. 4F is a schematic view of a balloon assembly having an interlaced jacket including mesh filaments.

FIG. 4G is a schematic view of a balloon assembly having an interlaced jacket including non-woven filaments.

FIG. 5A is a schematic view of another example of a balloon assembly including an interlaced jacket provided along an element body of the balloon assembly.

FIG. 5B is a schematic view of an additional example of a balloon assembly including an interlaced jacket proximate at least one nose of the balloon assembly.

FIG. 6A is a schematic view of another example of a balloon assembly including an interlaced jacket having varied filament densities.

FIG. 6B is a schematic view of an additional example of a balloon assembly including an interlaced jacket having varied filament densities.

FIG. 7 is a side view of a balloon assembly having an interlaced jacket, the balloon assembly including a blunt nose and a tapered nose.

FIG. 8 is a side view of another example of a balloon assembly having an interlaced jacket and at least one blunt nose including a terraced profile.

FIG. 9 is a cross-sectional one example of a balloon assembly including a multiple cellular balloon.

FIG. 10 is a cross-sectional another example of a balloon assembly including a multiple cellular balloon.

FIG. 11 is a side view of one example of a balloon assembly including one or more helical balloons.

FIG. 12A is a first cross-sectional view of one example of a balloon assembly including a compliant interior balloon and a structural exterior balloon.

FIG. 12B is a second cross-sectional view of the balloon assembly of FIG. 12A.

FIG. 13A are cross-sectional views of one example of a balloon assembly including a compressible substrate in a deployed configuration.

FIG. 13B are cross-sectional views of the balloon assembly of FIG. 13A in a compressed configuration.

FIG. 13C is a cross-sectional view of the balloon assembly of FIG. 13A in a compressed configuration.

FIG. 14 is a detailed schematic view of one example of a distal nose of a balloon assembly including the interlaced jacket bonded along a terraced profile.

### DETAILED DESCRIPTION

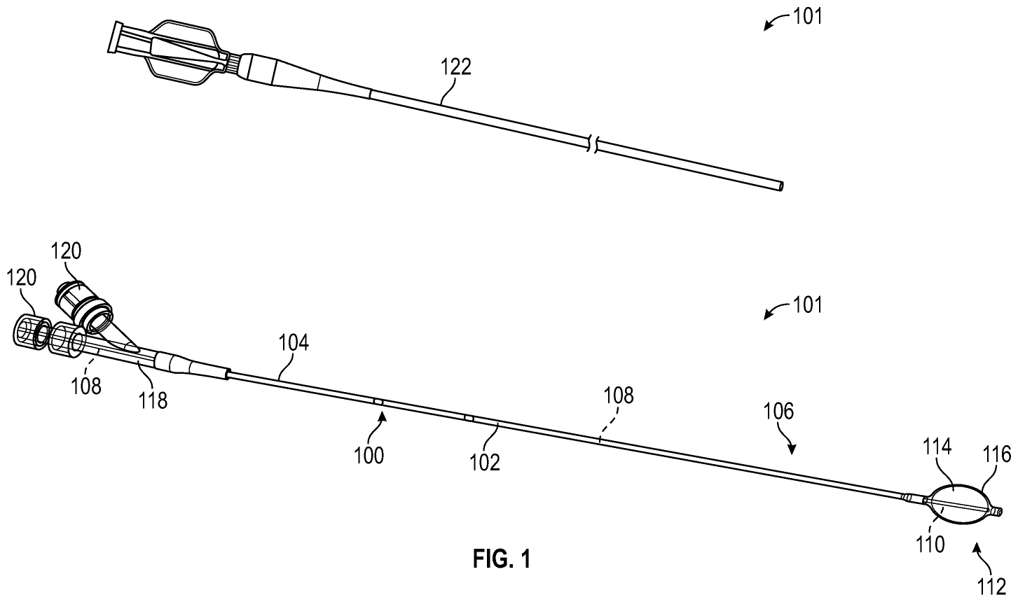

FIG. 1 shows one example of a balloon catheter assembly 101 (e.g., a balloon occlusion catheter assembly). The balloon catheter assembly 101 includes a balloon catheter 100. In other examples the balloon catheter assembly 101 includes or is used with other components, such as, but not limited to a guide sheath 122, one or more dilators, guidewires, additional introducers, guide sheaths, or the like. The balloon catheter assembly 101 including one or more components is in one example delivered to clinicians as a kit or other multi-component assembly configured for a single use or reuse of one or more of the components of the kit. As used herein, a balloon includes, but is not limited to, a balloon membrane, bladder or inflatable member having one or more of compliant, semi-compliant or non-compliant (pliable) materials, as well as foam or reticulated substrates or the like configured to expand and retract with the application or evacuation of a fluid (e.g., a gas or liquid) from the balloon.

Referring again to FIG. 1, the balloon catheter 100 includes a catheter shaft 102 extending between a proximal end portion 104 and a distal end portion 106. As further shown, the proximal end portion 104 is in one example coupled with a hub 118. The hub 118 optionally includes one or more hub ports 120 configured to provide one or more of access to the interior of the catheter shaft 102 or the delivery of fluids or aspiration of fluids through the catheter shaft 102 (including but not limited to the delivery and evacuation of inflation fluids to one or more balloon assemblies, such as the balloon assembly 112 shown in FIG. 1).

As further shown in FIG. 1, the balloon catheter 100 (e.g., a balloon catheter in this example) includes an occlusion assembly, such as a balloon assembly 112, coupled proximate to the distal end portion 106 of the catheter shaft 102. In one example, the catheter shaft 102 extends beyond the balloon assembly 112 for instance to an atraumatic tip. In FIG. 1, the balloon assembly 112 is shown in a deployed configuration, for instance, with a balloon membrane 114 including a jacket 116 (further described herein) in a deployed configuration. The catheter shaft 102. In one example includes an inflation port 110 extending through the side wall of the catheter shaft 102 and placing an inflation lumen 108 in communication with the interior of the balloon membrane 114. The introduction or evacuation of fluids (e.g., saline, gases or the like) into and from the balloon membrane 114 transitions the balloon membrane 114 between the deployed configuration shown in FIG. 1 and a compressed or stowed configuration. In the compressed configuration the balloon membrane 114 is withdrawn and gathered around the catheter shaft 102, for instance, for delivery of the balloon assembly 112 through the guide sheath 122 (or guide catheters, introducer sheaths or the like). FIG. 1 shows an inflation lumen 108 extending through the catheter shaft 102. In other examples, the balloon catheter 100 optionally includes a plurality of inflation lumens 108, for instance, inflation lumens configured for communication with one or more balloon membranes (e.g., proximal and distal balloons, an interior balloon nested within an exterior balloon or the like). The inflation lumens 108 in such an example facilitate the selective inflation and deflation of each of the balloon membranes to accordingly deploy the balloon assembly 112 (e.g., an occlusion assembly) as desired. In still other examples, delivery lumens are provided in the catheter shaft 102 to facilitate delivery of tools, instruments, or fluids into the vasculature, cavities or the like of the anatomy.

As previously described, the balloon catheter 100 includes one or more balloon assemblies 112 coupled along the catheter shaft 102. The balloon assembly 112 includes at least one balloon membrane 114 coupled with a jacket 116. In one example, the jacket 116 includes an interlaced jacket including interlaced filaments provided along the balloon membrane 114 to strengthen the balloon membrane 114 during inflation while at the same time constraining inflation of the balloon membrane 114 to a specified balloon profile. The interlaced filaments of the jacket 116 are in one example arranged in a configuration that diverges relative to a longitudinal axis of the catheter shaft 102. For example, the filaments of the jacket 116 are oriented at angles relative to the longitudinal axis of the catheter shaft 102 or the longitudinal axis of the balloon membrane 114. The interlaced filaments in these orientations are not aligned with the longitudinal axis of the catheter (e.g., the catheter shaft or the balloon membrane along the catheter shaft), and instead diverge from and converge toward the longitudinal axis. Orienting of the filaments of the jacket 116 in this way maintains structural support to the balloon membrane 114 while at the same time maintaining the flexibility of the overall balloon assembly including the balloon membrane 114. Accordingly, the balloon assembly 112 (e.g., the occlusion assembly) including the jacket 116 as well as the balloon membrane 114 can be inflated to a specified balloon profile while doing so at high pressures, for instance at 20 atmospheres or more (e.g., at 40 atmospheres or more, 60 atmospheres or more, or the like). Additionally, because the filaments of the jacket 116 are oriented at angles that diverge relative to the catheter shaft 102, the balloon assembly 112 provides an enhanced degree of flexibility compared to previous balloon assemblies 112 having longitudinally extending filaments extending along or substantially along the axis of the catheter shaft, the longitudinal axis of the balloon membrane or the like.

As further described herein, the balloon assembly 112 includes, but is not limited to, a compliant balloon, a semi-compliant balloon or a non-compliant balloon (such balloons are optionally constructed with, but not limited to, polyimide, polyurethane, poly ether block amide, PEEK, a composite of multiple polymers or the like). Because the jacket 116 is coupled with the balloon membrane 114, even with semi-compliant and compliant balloons inflated to high pressure such as 20 atmospheres or more, the jacket 116 maintains the balloon assembly 112 in the specified balloon profile. The compliant character of the semi-compliant balloon and elastic character of the compliant balloon facilitate the even distribution of pressures along the jacket 116 as the balloon membrane 114 deflects and deforms relative to the jacket 116. This provides even pressure distribution along the jacket 116, minimizes stress risers in the balloon membrane and minimizes blowouts, unpredictable expansion of the balloon membrane 114 (e.g., outside of the specified balloon profile) or the like.

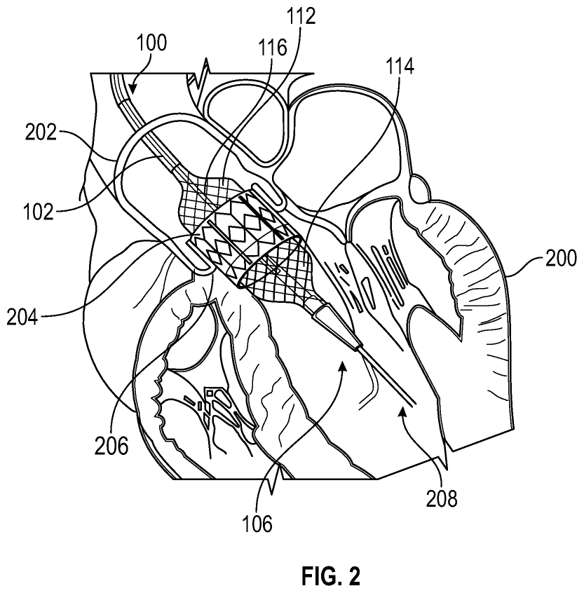

FIG. 2 shows the balloon catheter 100 of the balloon catheter assembly 101 in an operative configuration, for instance, positioned within the vasculature of a patient. In the example shown in FIG. 2, the balloon catheter 100 is navigated through a vessel 202, such as through the aorta proximate the heart 200. The balloon assembly 112 of the balloon catheter 100 is positioned within the existing valve 206 at the juncture between the aorta and the left ventricle of the heart 200. A replacement valve 204 is provided around the balloon assembly 112 (including for instance a jacket 116 as described herein and shown in FIG. 1). The replacement valve 204 is in an expanded configuration and ready for implantation at the existing valve 206. In one example, a guidewire 208 is navigated into the heart 200 over through one or more delivery or guide catheters (e.g., components of the balloon catheter assembly 101). The balloon catheter is passed along the guidewire 208 to position the balloon assembly 112 at the position shown in FIG. 2. When deployment of the balloon assembly 112 is desired, the balloon assembly 112 is optionally moved distally relative to a guide catheter to expose the balloon assembly 112. An inflation fluid is delivered through the inflation lumen 108 (see FIG. 1) and delivered through the inflation port 110 to inflate the balloon assembly 112 into the deployed configuration shown in FIG. 2.

When inflating the balloon assembly 112, for instance, to deploy and install the replacement valve 204, high pressure is optionally specified to fully deploy the balloon assembly 112 and the replacement valve 204 to an installation configuration that ensures the implanting of the replacement valve 204 at the existing valve 206 while at the same time minimizing collapse of the replacement valve 204 after withdrawal of the balloon assembly 112. With balloon assemblies reinforced with filaments substantially aligned with the balloon axis or catheter axis (local to the balloon) full deployment (e.g., dilation) of the replacement valve 204 with the balloon assembly at high pressures (20 atmospheres or more) in some circumstances causes significant pain or discomfort for the patient.

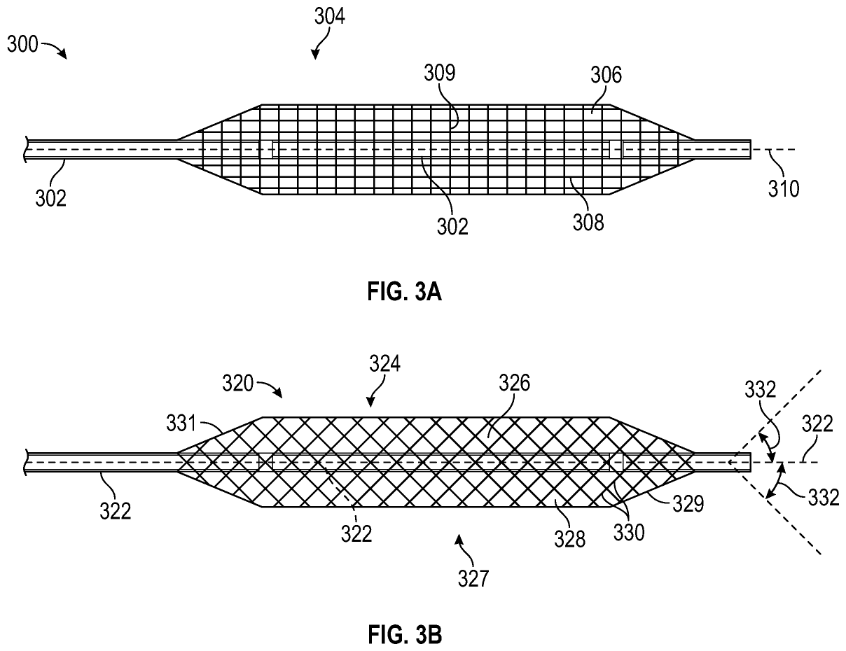

In contrast, the balloon assembly 112 includes a balloon membrane 114 and a jacket 116 (shown for instance with schematic filaments in FIG. 2) that constrains inflation of the balloon membrane 114 to a specified balloon profile (e.g., corresponding to the shape shown in FIG. 2) and also maintains a degree of flexibility greater than balloon assemblies having filaments substantially aligned with the balloon axis. In one example, as previously described herein, the balloon assembly 112 includes an interlaced jacket including one or more interlaced filaments that are provided at diverging angles relative to a longitudinal axis of the catheter shaft 102, a longitudinal axis of the balloon membrane 114 or the like. The interlaced filaments at diverging angles (one example of a diverging angle 332 is shown in FIG. 3B) orients the structural support of the balloon assembly 112 along axes that are not aligned with the longitudinal axis of the balloon assembly 112. Because the structural support is oriented at angles relative to the catheter shaft 102 the balloon assembly 112 maintains flexibility while in the deployed configuration. The jacket 116 maintains the flexibility of the balloon membrane 114 even when inflated to high pressures while at the same time providing structural support to the balloon membrane 114 to ensure inflation to the specified balloon profile (for instance to deploy the replacement valve 204 in a reliable manner).

FIGS. 3A and 3B show two examples of balloon catheters 300, 320. Referring first to FIG. 3A, the balloon catheter 300 includes a balloon assembly 304 coupled with a catheter shaft 302. The balloon assembly 304 includes a balloon membrane 306 and a plurality of aligned filaments 308 aligned parallel with one or more axes, such as the catheter shaft axis 310 shown extending along the catheter shaft 302. As further shown in FIG. 3A, the catheter shaft axis 310 corresponds with a longitudinal axis of the balloon membrane 306. The balloon assembly 304 optionally includes additional filaments such as circumferential filaments 309 extending around the perimeter of the balloon assembly 304 in a direction perpendicular to the catheter shaft axis 310.

As shown in FIG. 3A, the balloon assembly 304 is in a deployed configuration. The balloon membrane 306 is coupled with the aligned filaments 308, and the aligned filaments 308 are aligned (e.g., substantially parallel at least in part) with the catheter shaft axis 310. Specifically, the aligned filaments 308 extending from a proximal portion of the balloon catheter 300 to a distal portion are aligned (including parallel or substantially parallel) to the catheter shaft axis 310. As the balloon membrane 306 is inflated into the configuration shown in FIG. 3A the aligned filaments 308 extend along the catheter shaft axis 310 to provide support to the balloon membrane 306.

As previously described, the balloon membrane 306 is inflated in at least one example with high pressure fluid (e.g., 20, 40, 60 or more atmospheres). With inflation of the balloon membrane 306 to a high pressure, the aligned filaments 308 extending along the catheter shaft axis 310 cooperate with the balloon membrane 306 to provide one or more component supports extending along the length of the balloon assembly 304. In at least some examples, the balloon membrane 306, when positioned within the vasculature of the patient, in combination with the aligned filaments 308 (in contrast to diverging filaments as described herein) is substantially inflexible and accordingly may cause pain and discomfort to the patient. The aligned filaments 308 act as structural or columnar supports to the balloon membrane 306 and can resist bending deflection of the balloon assembly 304, for instance, between the proximal and distal ends of the balloon membrane 306. Accordingly, deformation, such as bending of the balloon assembly 304, radial deformation or the like is resisted by the aligned filaments 308. Instead, the balloon membrane 306 with the aligned filaments 308 forming a cage therearound has a rigid character.

In some examples, the inclusion of aligned filaments 308 is specified, for instance to provide a balloon assembly 304 having enhanced support characteristics that maintains its profile (shape, size or the like) when deployed. For instance, the deployed balloon assembly 304 of FIG. 3A includes enhanced rigidity that facilitates maintenance of a specified profile. In still other examples, the aligned filaments 308 are used in combination with diverging interlaced filaments (described herein) to modulate the flexibility of the balloon assembly. For instance, aligned filaments are used along portions of the balloon assembly configured to deployment in relatively straight vasculature or cavities, or where a rigid structure is specified. In contrast, diverging filaments are used along other portions of the balloon assembly specified for flexibility, for instance to conform to tortuous or non-linear vasculature or cavities. Accordingly, portions of the balloon assembly having aligned filaments 308 (e.g., as shown in FIG. 3A) have enhanced structural characteristics, such as rigidity, in comparison to portions of the assembly with diverging filaments specified for enhanced flexibility.

FIG. 3B shows another example of the balloon catheter 320. In this example, the balloon catheter 320 includes a balloon assembly 324 coupled with a catheter shaft 322. The balloon assembly 324 includes a balloon membrane 326 having a balloon body 327, a balloon distal nose 329, and a balloon proximal nose 331. The balloon catheter 320 includes a jacket, such as an interlaced jacket 328. The interlaced jacket 328 includes a plurality of interlaced filaments 330 extending at one or more diverging angles relative to the catheter shaft axis 322, also shown in FIG. 3B. For instance, each of the interlaced filaments 330 diverges relative to the catheter shaft axis 322 as shown with the diverging angles 332.

In one example, the jacket 328 is continuously coupled with the balloon membrane 326, for instance between the balloon proximal nose 331 and the balloon distal nose 329. In another example the jacket 328 is coupled with the balloon membrane 326 at one or more locations between the balloon proximal and balloon distal noses 331, 329. For instance, the jacket 328 is coupled at one or more bonding locations including, but not limited to, spot welds, localized bonds, adhesive interfaces or the like and includes decoupled spaces between bonding locations. In still other examples, the balloon membrane 326 is coupled with the jacket 328 at locations proximate to the balloon proximal nose 331 and the balloon distal nose 329. For instance, where crimping, bonding or the like is conducted between the balloon membrane 326 and the catheter shaft 322 in at least one example the jacket 328 is also bonded with one or more of the catheter shaft 322 and the balloon membrane 326 at those locations. Accordingly, the decoupling space between the bonding locations extends from the balloon proximal nose 331 to the balloon distal nose 329.

In another example, the jacket 328 is coupled with the balloon membrane 326 at one or more locations between the balloon proximal nose 331 and the balloon distal nose 329. For instance, the balloon membrane 326 is adhered to the jacket 328 dining construction of the assembly. The balloon membrane 326 receives a coating, spray application or the like of a bonding agent, such as an adhesive or dispersion prior to coupling of the jacket 328. Conversely, the interlaced filaments 330 of the jacket 328 are coated, dipped, or the like in a bonding agent prior to coupling with the balloon membrane 326 (e.g., prior to or as part of interlacing). In still other examples, the bonding agent is provided on the inside of the jacket 328, and as the jacket is applied or moved over top of the balloon membrane 326 the jacket 328 and the balloon membrane 326 are bonded together. In other examples, the bonding agent includes a dispersion, such as polyurethane or the like, applied to the balloon assembly 324 after assembly of the jacket 328 to the balloon membrane 326. The dispersion infiltrates through the jacket 328 (e.g., interstitial spaces between interlaced filaments 330) and bonds the jacket 328 along the balloon membrane 326. In some examples either the adhesive, dispersion or the like are locally applied to one or more locations (e.g., bonding locations) between or along the balloon membrane 326 and the jacket 328. Accordingly, with the bonding agents and techniques described herein the jacket 328 is coupled at one or more locations to the balloon membrane 326. In still other examples, the bonding agent is applied continuously along the jacket 328 and the balloon membrane 326 to facilitate the continuous coupling of the jacket 328 along the membrane 326.

As previously described and shown in FIG. 3B, the balloon assembly 324 includes one or more interlaced filaments 330 extending at diverging angles 332 relative to a catheter shaft axis 322 (e.g., corresponding to the balloon axis). The interlaced filaments 330 of the jacket 328 are provided at diverging angles 332 to misalign the interlaced filaments 330 relative to the catheter shaft axis 322. The misalignment of the interlaced filaments 330 provides a robust structural component around the balloon membrane 326 to facilitate the inflation of the balloon membrane 326 to a specified balloon profile while at the same time maintaining a degree of flexibility for the balloon assembly 324 (in comparison to assemblies having filaments aligned with a catheter shaft axis, as shown in FIG. 3A). For instance, with the balloon assembly 324 inflated to pressures of 20 atmospheres, 40 atmospheres, 60 atmospheres or the like, the balloon membrane 326 is deployed to the specified balloon profile shown in FIG. 3B while the interlaced jacket 328 supports the balloon membrane 326 (e.g., a semi-compliant, compliant or non-compliant membrane) in the specified balloon profile. Additionally, because the interlaced filaments 330 diverge from the catheter shaft axis 322 the structural support provided by the interlaced filaments 330 is directed along the diverging angle 332 and is thereby misaligned with the catheter shaft axis 322. The balloon assembly 324 retains a measure of flexibility relative to the balloon assembly 304 shown in FIG. 3A having the filaments 308 aligned with the catheter shaft axis 310.

In one example, the balloon assembly 324 of FIG. 3B deflects more (e.g., is more flexible when under stress) than the balloon assembly 304 shown in FIG. 3A because of the misalignment of the interlaced filaments 330 relative to the catheter shaft axis 322 in the balloon assembly 324. Because a rigid longitudinally extending cage is not provided around the catheter shaft axis 322 (as is provided around the balloon assembly 304 shown in FIG. 3A), the balloon assembly 324 —including the membrane and the jacket—deflects between the balloon proximal nose 331 and balloon distal nose 329. In an example, if the balloon distal nose 329 is rotated relative to the balloon proximal nose 331 (e.g., upwardly or downwardly relative to the page) each of the interlaced filaments 330 at an angle relative to the catheter shaft axis 322 more readily bends, deflects or the like as compared to the bending that occurs to the longitudinally aligned filaments 308 of the balloon assembly 304 shown in FIG. 3A when subject to the same rotational force. For instance, the angled filaments 330 enhance flexibility in one example by increasing the angle relative to the longitudinal axis for the filaments along an interior face of the bend (inside portion of the bend) and decreasing the angle relative to the longitudinal axis for filaments along an exterior face of the bend (outside portion of the bend). In contrast, longitudinally extending filaments, without or with a minimal angle to the longitudinal axis, remain along the longitudinal axis with bending and accordingly constrain bending, deflection or the like.

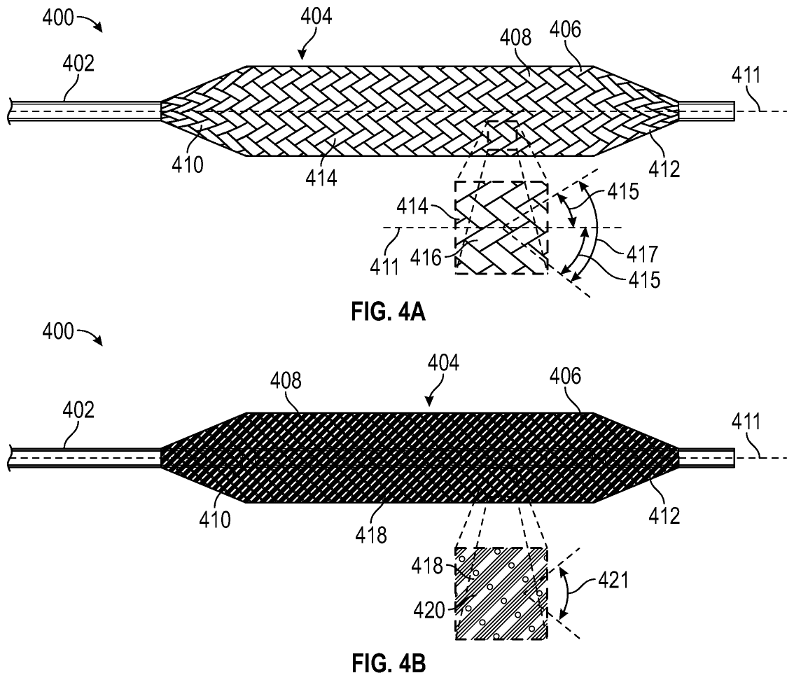

FIG. 3B shows one example of an interlaced jacket 328 including interlaced filaments 330 (presented schematically for ease of discussion) having a first pattern or arrangement of filaments to illustrate the orientation of the interlaced filaments 330 at diverging angles 332 relative to the catheter shaft axis 322. FIGS. 4A-4G described hereinbelow provide other examples of the balloon catheter 320 including a variety of interlaced filaments of interlaced jackets that orient the interlaced filaments at one or more diverging angles relative to the catheter shaft axis 322. Each of these examples facilitates the inflation of the balloon assembly, such as the balloon assembly 324 shown in FIG. 3B, under high pressure to a specified balloon profile while at the same time maintaining flexibility compared to the rigid structure formed with a balloon assembly having aligned filaments, such as the balloon assembly 304 shown in FIG. 3A. The various interlaced jackets shown in FIGS. 4A-G include, but are not limited to, interlaced jackets having braided filaments, woven filaments, knit filaments, crocheted filaments, nalbound filaments, mesh filaments, nonwoven filaments or the like.

Each of these interlaced jackets is optionally positioned along the entirety of the balloon assemblies, such as the balloon membrane, for instance along the balloon body, the balloon proximal nose, the balloon distal nose or the like. In other examples the interlaced jackets are provided at one or more locations including each of one or more of the balloon proximal and distal noses, the balloon body or the like.

In still other examples, the interlaced filaments are provided at various filament densities or varied interlacing patterns at differing locations along the balloon membrane to achieve specified support or flexibility characteristics for the balloon assembly. For instance, in one example filament densities are increased at one or more of the balloon proximal or distal nose relative to a lower second filament density along the balloon body. In still other examples the balloon density is reversed with the second filament density along the balloon body greater than the filament density at one or more of the balloon proximal or distal noses. In other examples, the interlacing pattern is changed in the jacket at one or more locations to achieve specified support or flexibility characteristics (tensile strength, radial strength, flexibility or the like).

FIGS. 4A-4G show various examples of a balloon catheter 400, for instance as a component of a balloon catheter assembly, such as the balloon catheter assembly 101 shown in FIG. 1. As previously described, the balloon catheters described herein include a jacket, such as an interlaced jacket, including one or more interlaced filaments provided at diverging angles relative to a catheter shaft axis 411.

Referring first to FIG. 4A, one example of the balloon catheter 400 is shown including an interlaced jacket 414 having a plurality of interlaced filaments 416 in a braided pattern. The balloon catheter 400 includes a catheter shaft 402 extending from a proximal end portion to a distal end portion. In the example of FIG. 4A, a distal end portion of the balloon catheter 400 is shown with the balloon assembly 404 coupled with the catheter shaft 402. The catheter shaft 402 includes one or more inflation lumens and one or more inflation ports configured to provide a flow of fluid (e.g., gas or liquid) to the balloon assembly 404 for inflation of the balloon membrane 406 between a deployed configuration and a compressed (or stowed) configuration.

As further shown in FIG. 4A, the balloon assembly 404 optionally extends between a balloon proximal nose 410 and a balloon distal nose 412. Each of the balloon proximal and distal noses 410, 412 are optionally coupled with the catheter shaft 402 using one or more features including, but not limited to, shrink tubing, welds, adhesives, crimping or the like. As further shown in FIG. 4A, the balloon membrane 406 includes a balloon body 408 extending between each of the balloon proximal nose 410 and the balloon distal nose 412.

The balloon assembly 404 (e.g., an occlusion assembly) including the balloon membrane 406 further includes an interlaced jacket 414. In the example jacket shown in FIG. 4A, the jacket 414 includes one or more interlaced filaments 416. As shown in the zoomed-in detail (provided below the initial view in FIG. 4A), the interlaced filament 416 includes a plurality of filaments provided in a braided pattern. The interlaced filaments 416 of the braid cooperate with one another, for instance with each of the filaments extending in opposed directions along the arms of an interlaced angle 417, to provide a robust structural support to the balloon assembly 404. The interlaced filaments 416 cooperate with one another to provide structural support around the balloon membrane 406 and ensure deployment of the balloon membrane 406 of the balloon assembly 404 to the specified balloon profile.

As shown, each of the interlaced filaments 416 are at a diverging angle 415 relative to the catheter shaft axis 411 (as previously shown in FIG. 3B). Further, the interlaced filaments 416 are provided at the interlaced angle 417 relative to other filaments extending in different directions. In one example, the interlaced angle 417 is the composite of the diverging angles 415 of each of the filaments 416 relative to the catheter shaft axis 411. With the braided configuration of the interlaced jacket 414, the interlaced filaments 416 are in one example braided at divering angles 415 between 30 and 80 degrees, 40 and 70 degrees, 50 and 60 degrees or the like. In the example of FIG. 4A, the interlaced filaments 416 are provided at an interlaced angle 417 of approximately 120 degrees, and corresponding diverging angles 415 relative to the catheter shaft axis of approximately 60 degrees.

The interlaced jacket 418 shown with the balloon catheter 400 in FIG. 4B includes another example of interlaced filaments 420. In this example, the interlaced filaments 420 are provided in a woven configuration, for instance with warp and weft filaments extending at approximately 90 degrees relative to each other. The interlaced angle 421 between the warp and weft filaments is approximately 90 degrees in the example shown. The interlaced filaments 420 forming the woven interlaced jacket 418 diverge from the catheter shaft axis to provide structural support and maintain flexibility of the balloon assembly 404 while inflated with relatively high pressure fluids, for instance at pressures of 20 atmospheres or more. Accordingly, each of the balloon catheter 400 examples shown in FIGS. 4A and 4B provide robust structural support to the balloon membrane 406 ensuring deployment to a specified balloon profile (e.g., specified size, shape or the like) while at the same time maintaining flexibility for the balloon assembly 404. This differs from the configurations that include longitudinally extending aligned filaments (e.g., as shown in FIG. 3A).

Referring again to FIG. 4B, in contrast to the embodiment shown in FIG. 4A, the interlaced jacket 418 shown in FIG. 4B includes woven interlaced filaments 420. Each of the woven interlaced filaments extend along separate arms of the interlaced angle 421 independently. Each of the filaments 420 provides resistance to deformation of the balloon membrane 406 during inflation of the balloon assembly 404. Stated another way, the interlaced filaments 420 of the woven interlaced jacket 418 each provide independent structural support to the balloon membrane 406 in their respective directions. The braided interlaced filaments 416 of the interlaced jacket 414 shown in FIG. 4A cooperate and interact with one another (according to the braid pattern) to support and constrain the deployment of the balloon assembly 404, for instance to the deployed configuration and the specified balloon profile shown in FIG. 4A. In one example, the interlaced filaments 420 of the woven interlaced jacket 418 of FIG. 4B are configured to operate independently of the balloon membrane 406. Optionally, the woven interlaced jacket 418 is coupled around the balloon membrane 406 without bonding of the jacket to the membrane. In another example, the interlaced jacket 416 shown in FIG. 4A—including the braided interlaced filaments 416—uses the balloon membrane 406 as an underlying substrate to support and maintain the braided configuration.

FIG. 4C shows another example of a balloon catheter 400 with a balloon assembly 404 having an interlaced jacket 422. In the example shown in FIG. 4C, the interlaced filaments 424 include one or more knit patterns. As with the previous examples, the interlaced filaments 424 of the knit interlaced jacket 422 extend at diverging angles relative to the catheter shaft axis 411 (as shown in the zoomed-in detail). The interlaced jacket 422 including the interlaced filaments 424 provides robust support to the balloon membrane 406 of the balloon assembly 404 (e.g., an occlusion assembly) including constraint of inflation of the balloon membrane 406 to a specified balloon profile while at the same time maintaining flexibility of the balloon assembly 404.

One example of a knit pattern is shown in FIG. 4C in the zoomed-in detail. The interlaced filaments 424 include filaments that double back or knot relative to one another to form one or more knit patterns. Because of the knotting, or doubling back, of the filaments, the interlaced jacket 422 provides structural stability to the balloon membrane 406 and the balloon assembly 404 even with a low density (e.g., 50 percent or less coverage of the balloon membrane 406 with the filaments 424, 428) of the interlaced filaments 424. Stated another way, the interlaced jacket 422 including the knotted (e.g., doubled back) filaments provides additional structural support to the balloon membrane 406 even with the interlaced filaments 424 in a spread configuration along the membrane 406. In contrast, the interlaced jackets shown in FIGS. 4A and 4B in examples include higher densities, for instance 50 to 100 percent coverage of the balloon membrane 406 with the filaments 416, 420.

FIG. 4D shows another example of the balloon catheter 400 including a balloon assembly having another pattern of interlaced filaments 428. In this example, the interlaced filaments 428 are in a crochet pattern. As with other examples described herein, the interlaced filaments 428 converge toward and diverge away from the catheter shaft axis 411 and are accordingly arranged at diverging angles relative to the catheter shaft axis 411. The interlaced filaments 428 thereby maintain flexibility in the balloon assembly 408 (the occlusion assembly) while at the same time providing structural support to the balloon membrane 406 and constraining the balloon membrane 406 to a specified balloon profile.

One example of the interlaced jacket 426 including at least one pattern of crocheted interlaced filaments 428 is shown in FIG. 4D. In a manner similar to the knit pattern of interlaced filaments 424 previously shown in FIG. 4C, the interlaced filaments 428 of the interlaced jacket 426 are knotted or doubled back over each other in a repeating pattern. Accordingly, the crocheted interlaced filaments 428 in one example have a low filament density relative to more tightly packed configurations (e.g., braids, weaves or the like). In one example, the interlaced jacket 426 (as well as the interlaced jacket shown in FIG. 4C) uses a lower density of interlaced filaments 428 to decrease the profile of the jacket along the balloon assembly 408.

FIG. 4E includes another example of the balloon catheter 400 having an interlaced jacket 430. As with previous examples, the interlaced jacket 430 is shown in the zoomed-in detail below the balloon assembly 404. The interlaced jacket 430 includes interlaced filaments 432 provided in another pattern. In this example, the interlaced filaments 432 are provided in a nalbound pattern that orients the interlaced filaments 432 at diverging angles relative to the catheter shaft axis 411 (as well as relative to the longitudinal axis of the balloon membrane 406). With the nalbound interlaced filaments 432, the filaments are stitched in contrast to the use of loops (e.g., in a crochet pattern). The interlaced jacket 430 including the nalbound interlaced filaments 432 is configured to constrain inflation of the balloon membrane 406 to the specified balloon profile and provide structural support to the balloon membrane 406 while inflated at relatively high pressures (e.g., 20 atmospheres or more).

Referring now to FIG. 4F, another example of an interlaced jacket 434 usable with the balloon catheter 400 is provided. In this example, the interlaced jacket 434 includes a mesh of interlaced filaments 436. The interlaced filaments 436 are shown in the zoomed-in detail and include a repeating mesh pattern. The interlaced filaments 436, as with the other embodiments described herein, extend at one or more angles that diverge relative to the catheter shaft axis 411. The interlaced jacket 434 provides a robust structural support to the balloon membrane while at the same time maintaining flexibility, for instance relative to longitudinally extending fibers shown in FIG. 3A. As shown in FIG. 4F, the interlaced filaments 436 are provided at a variety of angles relative to the catheter shaft axis 411 to maintain flexibility while at the same time supporting the balloon membrane 406 and constraining inflation of the membrane 406 into the deployed configuration at high pressures.

FIG. 4G shows another example of an interlaced jacket 438. In this example, the interlaced jacket 438 includes interlaced filaments 440 provided in a nonwoven pattern. For instance, the interlaced filaments 440 are spun into a matted configuration while in a fluid or glass transition state. The filaments 440 are bound in a non-woven pattern as the filaments 440 cool and solidify. Optionally, a polymerizing coating, bonding agent or the like is applied to the filaments 440 to initiate bonding between the filaments 440. Like the other examples described herein, the nonwoven interlaced filaments 440 are at diverging angles relative to the catheter shaft axis 411. By providing the interlaced filaments 440 at one or more angles relative to the catheter shaft axis 411 both flexibility and structural support are provided to the balloon assembly.

The interlaced jackets described herein are, in examples, provided along the entirety of the balloon assembly 404 for instance including each of the balloon distal and proximal noses 410, 412 the balloon body 408 and the like. In other examples, the interlaced jackets described herein are provided along one or more portions of the balloon assembly 404 including, but not limited to, one or more of the balloon body 408, one or more of the noses such as the balloon proximal nose 410 and the balloon distal nose 412 or combinations of two or more of these features.

The balloon membranes 406 of the various balloon assemblies 404 described herein are selected from one or more of compliant, semi-compliant or non-compliant balloon membranes. The interlaced jacket is provided over the balloon membrane 406. Optionally, the interlaced jacket 430 extends continuously around the balloon membrane 406. In another example, the interlaced jacket 430 extends around a portion of the balloon membrane 406 (e.g., a portion of the circumference). The interlaced jacket 430 provides robust structural support that limits the inflation of the balloon membrane 406 to the specified balloon profile while at the same time maintaining a degree of flexibility greater than that provided, for instance, by longitudinally extending filaments (see FIG. 3A).

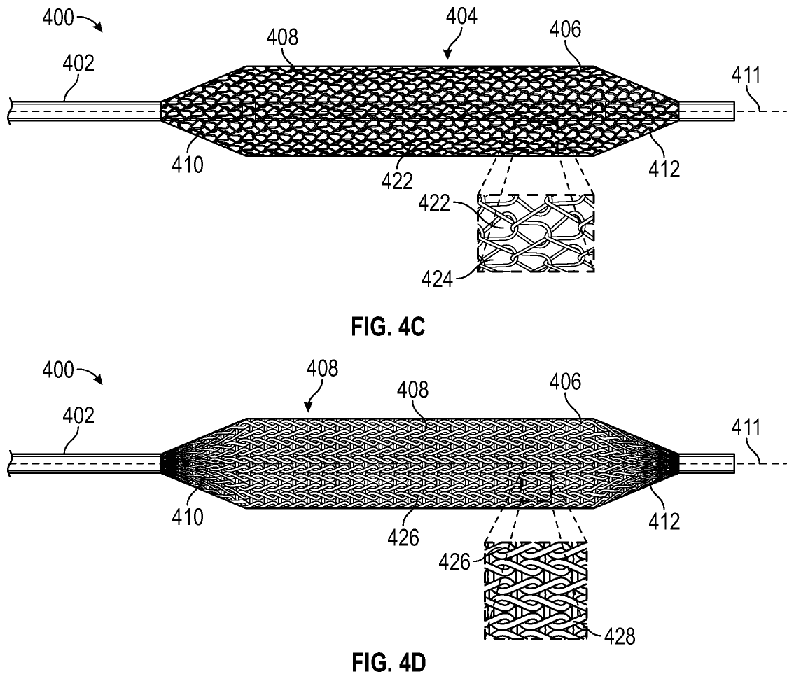

FIGS. 5A and 5B show examples of a balloon catheter 500 including jackets 514 at various locations along the balloon assembly 504. As previously described herein the jackets 514 are coupled with the balloon assembly 504 at one or more locations, for instance at or along one or more of the balloon proximal nose 508, balloon distal nose 510 or the balloon body 512. In other examples the interlaced jacket 514 is provided at two or more locations along the balloon catheter 500.

Referring first to FIG. 5A, the balloon catheter 500 includes a balloon assembly 504 coupled with the catheter shaft 502. The balloon membrane 506 of the assembly is surrounded in part by the jacket 514. As shown in FIG. 5A, the jacket 514 extends around the balloon body 512 and is proximate to each of the balloon proximal nose and balloon distal nose 508, 510. The jacket 514 includes one or more interlaced filaments extending at diverging angles relative to an axis, such as the catheter shaft axis of the catheter shaft 502 (e.g., corresponding to a longitudinal axis of the balloon membrane 506).

Referring again to FIG. 5A, the jacket 514 is provided along the balloon body 512 between the balloon proximal nose 508 and the balloon distal nose 510. In one example, the jacket 514 extends along the balloon body 512 and extends into or over at least a portion of the balloon proximal and distal noses 508, 510 (for instance into the transition between these features). The interlaced jacket 514 provides a support structure to the balloon membrane 506 and ensures the balloon body 512 (e.g., the portion of the balloon membrane having the largest diameter) deploys to a specified balloon profile for instance a specified diameter, shape or the like. With the balloon assembly 504 in the deployed configuration (shown in FIG. 5A) the balloon catheter 500 provides a balloon assembly 504, at a specified balloon profile constrained by the jacket 514.

The balloon membrane 506 is constructed with one or more of semi-compliant, non-compliant or compliant materials. In examples where the balloon membrane 506 is constructed with a semi-compliant or non-compliant material, the interlaced jacket 514 is provided along the balloon body 512 (and optionally absent in the balloon distal and proximal noses 510, 508) to constrain inflation of the balloon membrane 506 to the specified balloon profile (in this example a specified diameter). The balloon proximal and distal noses 508, 510 because of their construction with semi-compliant or non-compliant materials have a minimized risk for overinflation into a profile larger than the specified balloon profile and accordingly, in at least this example, the interlaced jacket 514 is localized to the balloon body 512. Additionally, by locating the interlaced jacket 514 along the balloon body 512 (the largest diameter portion of the balloon membrane 506) tapering of the interlaced jacket 514 and corresponding spreading or gathering of the filaments of the interlaced jacket 514 along the (tapered) balloon proximal and distal noses 508, 510 is minimized (e.g., substantially minimized or eliminated).

Where the balloon membrane 506 is constructed with a semi-compliant or compliant material (or even with a non-compliant material when operated at high pressures) the balloon proximal and distal noses 508, 510 are in some examples provided with one or more nose cones or other support features coupled around the balloon membrane 506 to constrain and support the inflation of the balloon membrane 506 into the specified balloon profile with operation of the balloon catheter 500 at high pressures, for instance pressures at 20 atmospheres or more (e.g., including 40 atmospheres, 60 atmospheres or more or the like). The optional nose cones or other support features are coupled with the balloon proximal nose 508 and the balloon distal nose 510 prior to installation of the interlaced jacket 514. In other examples the nose cones or other support features are coupled over the balloon proximal and distal noses 508, 510 at or after installation of the interlaced jacket 514. In these examples nose cones or other support features are optionally used to provide a joint or other coupling feature configured to extend along the proximal and distal ends of the interlaced jacket 514 and reliably organize and store the ends of the filaments of the jacket 514 to minimize fraying or delamination of the jacket from the balloon membrane 506.

Referring now to FIG. 5B, another example of the balloon catheter 500 is shown. In this example, the jacket 514 is localized to one or more of the balloon proximal nose 508 or the balloon distal nose 510. The interlaced jacket 514 is optionally absent from the balloon body 512. In other examples, the interlaced jacket 514 extends from the balloon distal nose 510 into at least a portion of the balloon body 512. Alternatively, the interlaced jacket 514 associated with the balloon proximal nose 508 extends along a portion of the balloon body 512. As shown in FIG. 5B, the interlaced jacket 514, when localized to the balloon proximal or distal noses 508, 510, provides a constraining support feature around each of the noses 508, 510. The interlaced jacket 514 is optionally coupled with the balloon distal nose 510 to provide a specified profile (e.g., included as part of the specified balloon profile) to the balloon distal nose 510. For instance, the interlaced jacket 514 is shaped by gathering or coupling of the interlaced filaments along the balloon distal nose 510 to provide a specified blunt shape to the balloon distal nose 510. In some examples the blunt shape is useful for one or more procedures including, but not limited to, valve replacement such as trans-aortic valve replacement (TAVR). In other examples, a tapered shape is provided at either or both ends of the balloon membrane 506. In such an example, the interlaced jacket 514 is coupled around the balloon proximal or distal noses 508, 510 and constrains inflation of the balloon membrane 506 at the noses 508, 510 into the specified tapered shape (e.g., a specified balloon profile). In still other examples, the balloon membrane or other base component of the balloon assembly (e.g., a foam substrate) is constructed with the specified blunt or tapered profile at one or more of the noses, and the interlaced jacket 514 at the noses enhances the maintenance of the specified profile or profiles including tapered profiles of the noses.

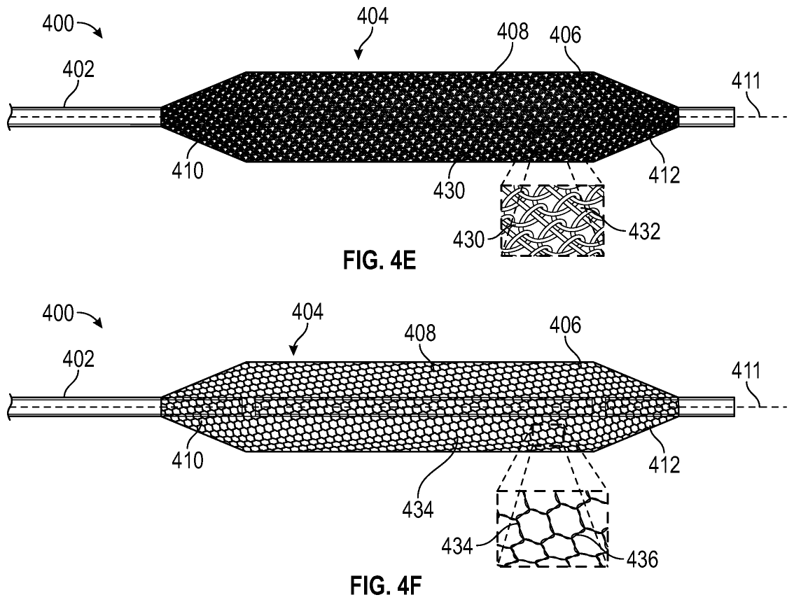

Referring now to FIGS. 6A and 6B, another example of a balloon catheter 600 is shown. Each of the variations of the balloon catheter 600 includes a balloon assembly 604. Referring first to FIG. 6A, the balloon assembly 604 includes a jacket 614 along a balloon body 608. The jacket 614 in this example is an interlaced jacket having a higher filament density along the balloon body 608 relative to filament densities of either or both balloon proximal or distal noses 610, 612. In contrast, the other example of the balloon catheter 600 shown in FIG. 6B includes a higher filament density in the balloon proximal and distal noses 610, 612 relative to a filament density in the balloon body 608.

Referring again to FIG. 6A, the balloon assembly 604 is shown coupled to a catheter shaft 602 in a manner similar to previous embodiments described herein. For instance, the balloon assembly 604 includes a balloon membrane 606 coupled with a catheter shaft 602. A jacket 614, such as an interlaced jacket including a plurality of interlaced filaments, is provided around the balloon membrane 606. The interlaced jacket 614 is optionally a continuous interlaced jacket 614 coupled around the circumference of the balloon membrane 606. In other examples, the interlaced jacket 614 extends around a portion of the balloon membrane 606 (e.g., includes one or more discontinuities, gaps or the like).

As further shown in FIG. 6A, the balloon body 608 including the interlaced jacket 614 thereon includes interlaced filaments 616 having a first filament density greater than a corresponding second filament density of the interlaced filaments 618 of the jacket 614 proximate to the balloon proximal and distal noses 610, 612. In one example, additional interlaced filaments 616 including one or more of increased pies per inch, pic count, pic angle, pitch, bundling of multiple parallel filaments or the like are provided in the balloon body 608 relative to the balloon proximal and distal noses 610, 612. Increased density of filaments in the balloon body 608 is optionally decreased in either or both of the proximal and distal noses (e.g., by decreasing pics per inch, pic count, pic angle, pitch, unbundling of parallel filaments or the like). By providing a decreased filament density at one or more of the balloon proximal or distal noses 610, 612, as the interlaced jacket 614 is installed along the balloon membrane 606, the jacket 614 readily tapers at each of the proximal and distal noses 610, 612. Because the filaments are less dense at the proximal and distal noses 610, 612, gathering, pleating, folding, or the like are minimized as filaments are conformed (e.g., shaped) along the noses.

Referring now to FIG. 6B, the balloon assembly 604 also includes the jacket 614 along each of the balloon body 608 and one or more of the balloon proximal and distal noses 610, 612. In contrast to FIG. 6A, the jacket 614 of FIG. 6B includes a second filament density, for instance of the interlaced filaments 618 associated with one or more of the balloon proximal and distal noses 610, 612, that is higher relative to a first filament density of the interlaced filaments 616 proximate to the balloon body 608.

The interlaced jacket 614 is in one example formed separately from the balloon membrane 606. For instance, the interlaced jacket 614 is braided, knit, woven or the like around a cylindrical mandrel and then positioned over the balloon membrane 606. As the interlaced jacket 614 is positioned over the balloon membrane 606, the higher density of filaments proximate one or more of the noses 610, 612 facilitates the tensioning of the interlaced jacket 614 and the tapering of the interlaced jacket 614 along the balloon distal and proximal noses 612, 610. The higher density of the interlaced filaments 618 at the proximal and distal noses 610, 612 allows for spreading of the dense filaments (e.g., during tensioning) along each of the noses 610, 612 as the interlaced jacket 614 is formed around each of the noses.

In another example, the filaments of the jacket 614 are interlaced along the balloon membrane 606 and bonded with the balloon membrane (e.g., with adhesives, dispersions or the like). The balloon assembly 604, including the membrane 606 and the interlaced jacket 616, is then bonded with the catheter shaft 602 (e.g., with bonding agents, welds, crimping, crimped rings or the like). For instance, the balloon assembly 604 is decoupled from a mandrel and coupled with the catheter shaft 602.

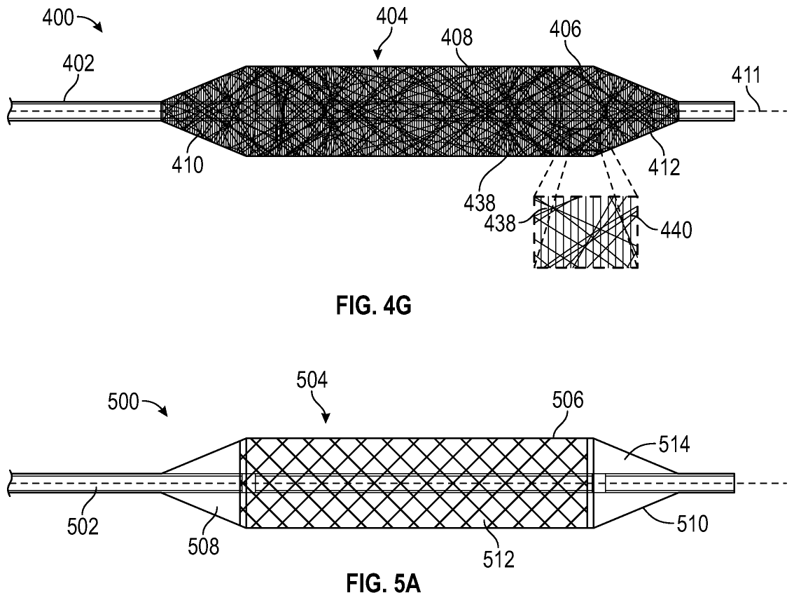

FIG. 7 shows another example of a balloon catheter 700 having a balloon assembly 704. In this example, the balloon assembly 704 includes a balloon membrane 706 or other deformable substrate and an interlaced jacket 714 extending around the balloon membrane 706. The interlaced jacket 714 extends over each of the balloon body 708, the balloon proximal nose 710 and the balloon distal nose 712. In other examples, as described herein, the interlaced jacket 714 extends over one or more of these features for instance one or more of the balloon distal nose 712, balloon proximal nose 710 or the balloon body 708.

The balloon distal nose 712 and the balloon proximal nose 710 have differing profiles. In this example, the balloon assembly 704 includes a blunt profile proximate the balloon distal nose 712 and a tapered profile proximate the balloon proximal nose 710. By varying profiles of one or more of the balloon proximal and distal noses 710, 712, the balloon catheter 700 is useful for certain procedures. For instance, with a trans-aortic valve replacement procedure (TAVR), a balloon distal nose 712 having a blunt profile localizes the balloon assembly 704 to the area corresponding to the aortic valve such as the existing valve 206 shown in FIG. 2. Variations in profile of either or both of the balloon distal nose 712 and the balloon proximal nose 710 are optionally provided by one or more features associated with the balloon assembly 704 (e.g., processing of the balloon membrane 706, shaping of the membrane with structural supports such as jackets or nose cones or the like).

In one example, the blunt or tapered profiles proximate the balloon distal nose 712 and the balloon proximal nose 710 are provided by the balloon membrane 706 (e.g., through molding, processing of the membrane or the like). In other examples, the variations in profile are provided, at least in part, by the jacket 714. For instance, the jacket 714 is formed on a mandrel having a specified shape including the specified profiles of the balloon distal nose 712 and the balloon proximal nose 710. The jacket 714 is interlaced (e.g., woven) along the corresponding mandrel to accordingly have a corresponding shape to the specified balloon profile including one or more of the blunt or tapered profiles for the balloon proximal and distal noses 710, 712. The jacket 714 is removed from the mandrel and installed along the catheter shaft 702. The substrate of the balloon assembly 704, for instance the balloon membrane 706, is deployed within the jacket 714 and bonded to the jacket 714. The jacket 714 formed with the desired specified profile constrains inflation of the balloon membrane 706 (including deployment of another substrate, such as a foam substrate described herein) to the specified balloon profile including, but not limited to, one or more of a blunt balloon distal nose 712 or a tapered balloon proximal nose 710.

In other examples, the membrane of the balloon assembly 704 is constrained by one or more other structural support features coupled with the balloon membrane 706. For instance, one or more pliable nose cones are coupled around the balloon distal nose 712 or the balloon proximal nose 710 to constrain inflation of the balloon membrane 706 into the specified balloon profile including one or more of blunt or tapered configurations at the noses 710, 712. In some examples, cones applied to one or more of the balloon distal nose 712 or the balloon proximal nose 710 are formed with semi-compliant or non-compliant materials. Accordingly, the balloon assembly 704 is constrained by the cones to deploy into a specified balloon profile including one or more of a blunt distal profile, tapered proximal profile, or other profiles at either or both of the balloon proximal or distal noses 710, 712. Nose cones include one or more of polyamide, polyurethane, poly ether block amide, PEEK, a composite of multiple polymers or the like. Optionally, the nose cones are integrally formed with the balloon membrane 706 (e.g., during molding) or adhered to the balloon membrane. In other examples, filaments including braided and unbraided polymer filaments are coupled with the proximal or distal noses 710, 712 to provide nose cones.

In one example, the balloon membrane 706 as described herein is constructed with a compliant membrane. The combination of the jacket 714 (e.g., an interlaced jacket) and one or more cones applied to the balloon distal nose 712 and the balloon proximal nose 710 constrains inflation of the compliant balloon membrane 706 to the specified balloon profile. In still other examples, the jacket 714 is coupled along one or more of the features of the balloon membrane 706, such as the balloon body 708 or one or more of the balloon proximal and distal noses 710, 712 without nose cones. In this configuration, the jacket 714 constrains inflation of the compliant balloon membrane 706 (including the noses 710, 712) to the specified balloon profile.

In other examples, the jacket 714 is optionally located or localized to the balloon body 708 and absent proximate the noses. With the jacket 714 absent from the noses 710, 712, complications such as gathering, spreading, wrinkling or the like of the interlaced filaments of the jacket 714 along the noses are minimized. In other examples, the jacket 714 extends over one or more of the cones applied to the balloon distal nose 712 or the balloon proximal nose 710 and cooperates with the balloon membrane 706 to anchor the cones to each of the balloon proximal and distal noses 712, 710.

In still other examples, where the jacket 714 extends over one or more of the balloon proximal nose 710 or the balloon distal nose 712, the jacket 714 including interlaced filaments forming the jacket are in one example provided in a woven pattern. The filaments in the woven pattern provide independent strength and resistance to spreading of the filaments that may otherwise occur with coupling of the jacket along a blunt or tapered profile such as the blunt profile of balloon distal nose 712 and the tapered profile of the balloon proximal nose 710. Accordingly, complications caused by spreading, gathering, wrinkling or the like are minimized with one or more of the interlaced configurations described herein including, but not limited to, the woven configuration.

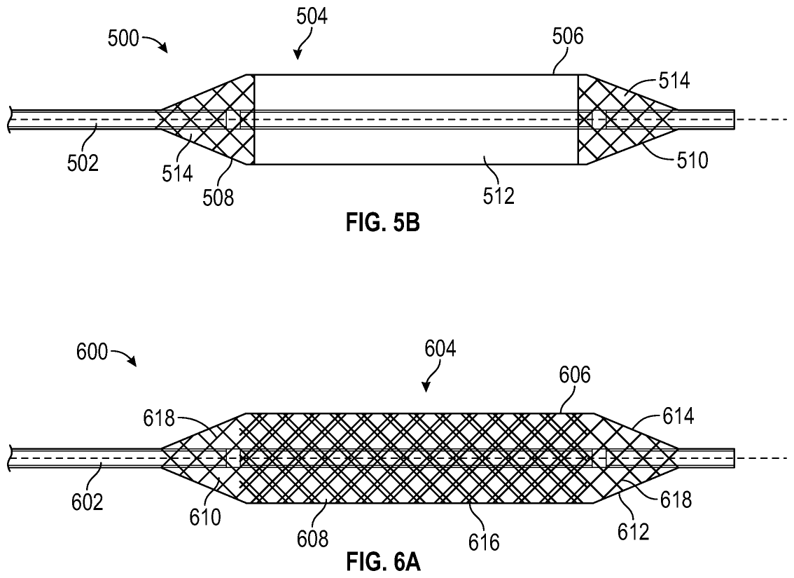

FIG. 8 is a detailed view of one example of a balloon distal nose such as the balloon distal nose 712. (shown in FIG. 7). In this example, the balloon membrane 706 includes a terraced profile 800. The terraced profile 800 includes a plurality of tapered surfaces 802 interposed between one or more anchor surfaces 804. As previously described, in some examples a blunt profile proximate the balloon distal nose 712 is desirable for one or more procedures using the balloon catheter assembly 704. Where the jacket 714 is applied over a blunt profile, such as the profile shown at the balloon distal nose 712, attachment of the interlaced filaments of the jacket 714 is difficult. Spreading, fraying, gathering, wrinkling or the like of the filaments of the jacket 714 may occur in the abrupt transition of the blunt profile from the large diameter of the balloon body 708 to the smaller diameter of the catheter shaft 702.

The terraced profile 800 provides a virtual blunt profile that minimizes (e.g., eliminates or minimizes) spreading, fraying, gathering, wrinkling or the like. The tapered surfaces 802 of the terraced profile 800 provide a composite blunt configuration, as shown in the larger view provided in FIG. 7. The anchor surfaces 804 provided at one or more locations along the terraced profile 800 provide anchor locations for the interlaced filaments of the jacket 714. With one or more bonding agents, such as adhesives, dispersions or the like applied to the jacket 714 and the balloon membrane 706, the filaments are anchored or bonded along the anchor surfaces 804 while at the same time extending along and conformed to the shape of the terraced profile 800. The interlaced jacket 714 is coupled with the blunt nose 712 without spreading, fraying gathering or wrinkling of the filaments while also constraining inflation of the balloon membrane 706 to the specified blunt profile.

The terraced profile 800 is in one example formed as part of the balloon during molding, processing of the balloon (e.g., mechanical deformation) or the like. For instance, a mandrel includes the terraced profile 800, and the balloon membrane 706 is formed along the terraced profile to shape the anchor surfaces 804 and the tapered surfaces 802. In other examples, one or more cones are applied to the balloon distal nose 712 (or proximal nose 710) having the terraced profile 800 shown in FIG. 8 (e.g., another example of a blunt profile). The interlaced jacket 714 is bonded over top of the terraced nose cone.

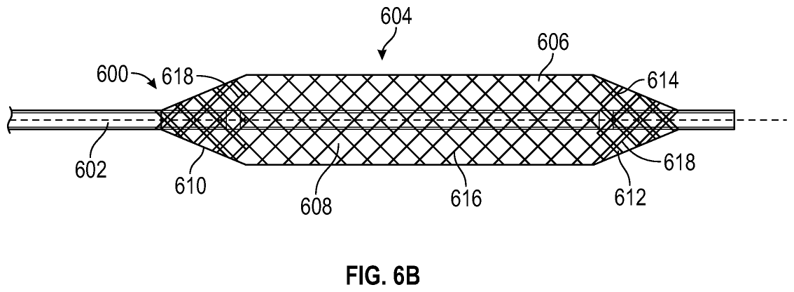

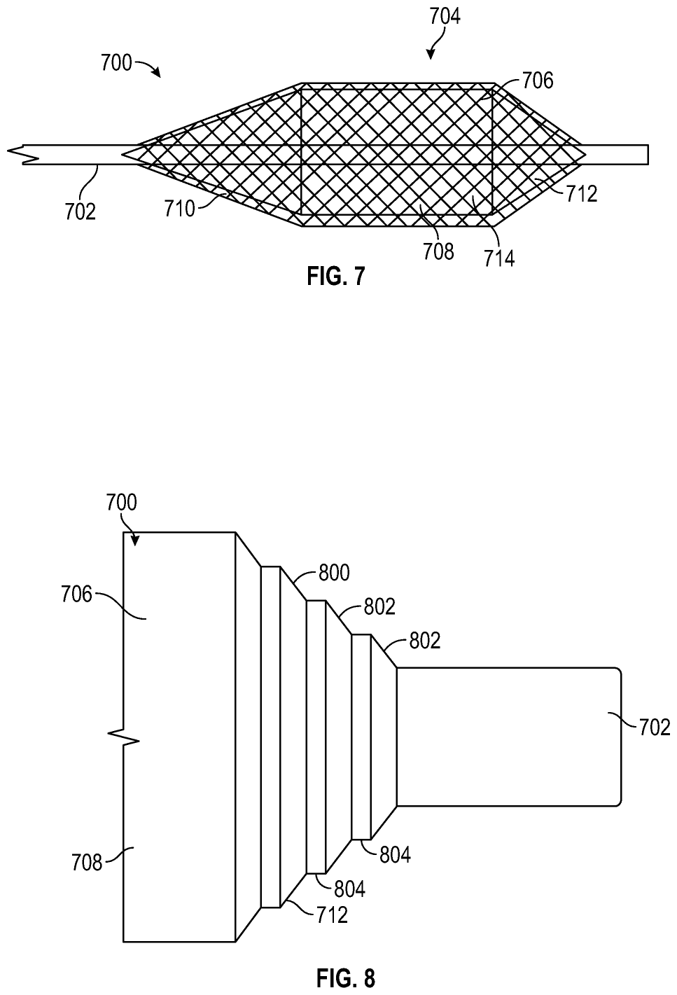

FIGS. 9 and 10 show two examples of a balloon assembly 904 formed with a plurality of balloon cells 906. Referring first to FIG. 9, the balloon assembly 904 includes one or more balloon cells 906 provided around the catheter shaft 902 The balloon cells 906 extend longitudinally along the catheter shaft 902, for instance proximate to a distal end portion of the catheter (such as the distal end portion 106 in FIG. 1). In another example, the balloon cells 906 extend helically around the catheter shaft 902 as well as longitudinally. The plurality of balloon cells 906 are provided in a distributed pattern around the catheter shaft 902. The balloon cells 906 cooperate to form the specified balloon profile for the balloon assembly 904 of the balloon catheter 900. In one example, each of the balloon cells 906 are coupled along the catheter shaft 902. For instance, the balloon cells 906 are coupled with the catheter shaft 902 with an adhesive, weld or the like extending between the catheter shaft 902 and one or more of the balloon cells 906.

In another example, the balloon assembly 904 includes a plurality of the balloon cells 906 joined together along one or more structural supports 908 in between each of the balloon cells 906. The structural supports 908 include one or more of struts, sidewalls, common sidewalk or the like shared between the balloon cells 906. In the example shown in FIG. 9, each of the balloon cells 906 includes a component sidewall joined with the adjacent component sidewall of another balloon cell 906. The structural supports 908 (in this example, common sidewalls) extend from the catheter shaft 902 toward an outer perimeter of the balloon assembly 904. The outer perimeter of the balloon assembly 904 in one example corresponds to the specified balloon profile for the balloon catheter 900. The common sidewalls or structural supports 908 of FIG. 9 cooperate with the remainder of the balloon cells 906 to maintain the balloon cells 906 and the overall balloon assembly 904 in the specified balloon profile when inflated. For instance, the structural supports 908 (e.g., the common sidewalls) cooperate to retain each of the balloon cells 906 in the inflated configuration shown in FIG. 9. Stated another way, the structural supports 908 anchor the exterior of each of the balloon cells 906 to the catheter shaft 902 and accordingly constrain inflation of the balloon cells 906 to the overall specified balloon profile formed by the composite assembly of the cells 906.

In another example, the balloon assembly 904 of the balloon catheter 900 includes one or more other structural supports in combination with the structural supports 908. In this example, a jacket 910 is coupled around the balloon cells 906 along the perimeter of the balloon assembly 904. The jacket 910 includes, but is not limited to, one or more of the interlaced jacket examples previously described herein. The interlaced jacket includes filaments extending at diverging angles relative to a catheter shaft axis to maintain flexibility in the balloon assembly 904 while also constraining and reinforcing the balloon cells 906 to the specified balloon profile. The jacket 910 and the other structural supports 908 cooperate to reinforce the balloon assembly 904 even while the balloon cells 906 are inflated with high pressure fluids for instance fluids supplied to the balloon cells 906 at 20 atmospheres, 40 atmospheres, 60 atmospheres or more.

In other examples, the jacket 910 includes a coating, sleeve or the like applied around the balloon cells 906 (e.g., a laminate, curable fluid or the like). In one example, the jacket 910 is a liquid applied to the balloon assembly 904 that surrounds each of the balloon cells 906 and fills gaps 912 between the component balloon cells 906. The jacket 910 closes the gaps 912 between each of the balloon cells 906 and the balloon assembly 904 maintains a smooth or cylindrical profile (e.g., the specified balloon profile).

FIG. 10 shows another example of the balloon catheter 900 including multiple cells. Each of the balloon cells 906 is co-extruded with the catheter shaft 902 or coupled with the catheter shaft 902 as the cells are extruded. For instance, the balloon cells 906 include but are not limited to one or more compliant, semi-compliant or non-compliant materials coextruded together to form the balloon cells 906 shown in FIG. 10. The balloon assembly 904 of FIG. 10 includes structural supports 908 between each of the cells 906, for instance common sidewalls formed during the co-extrusion of each of the balloon cells 906.

As further shown in FIG. 10, the balloon catheter 900 optionally includes a jacket 910. The jacket 910 includes, but is not limited to, an interlaced jacket constructed with one or more interlaced filaments provided around the balloon cells 906. In another example, the jacket 910 includes a coating, sleeve or the like applied over the balloon cells 906. The coating fills one or more gaps 912 between each of the balloon cells 906.

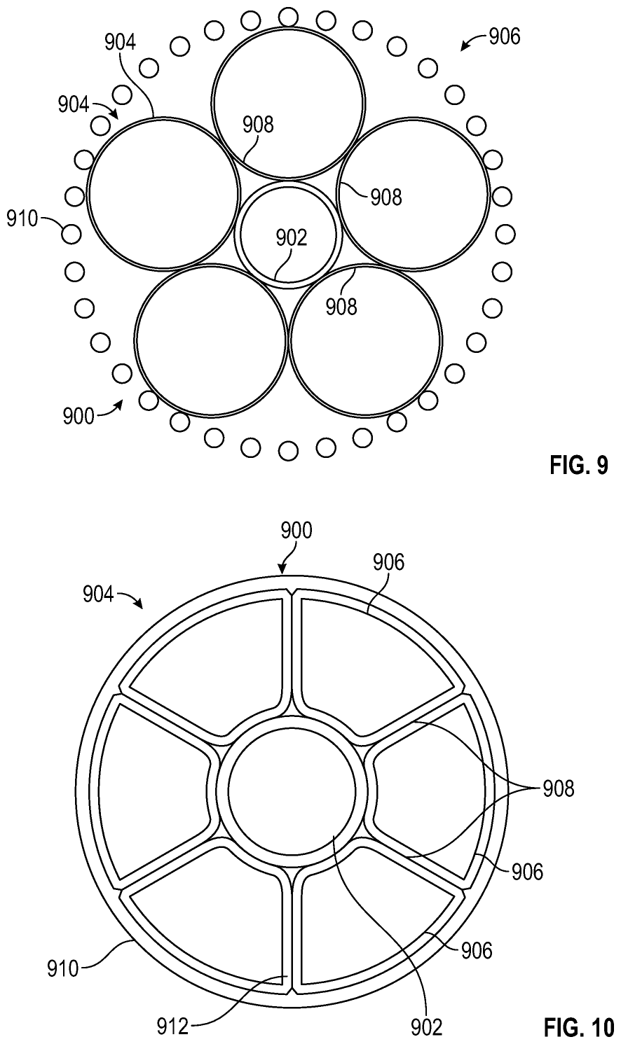

FIG. 11 shows another example of a balloon catheter 1100 including a balloon assembly 1104. The balloon assembly 1104 includes at least one balloon membrane 1106 in a helical configuration along the catheter shaft 1102 that corkscrews along the catheter shaft 1102 between the balloon proximal nose 1110 and the balloon distal nose 1112. The balloon body 1108 formed by the balloon membrane 1106 has a near isodiametric configuration. In another example, the balloon catheter 1100 includes a two or more balloon membranes 1106 helically wound along the catheter shaft 1102.

Optionally, the balloon membrane 1106 includes structural supports in a manner similar to the multi-cellular balloons shown in FIGS. 9 and 10. For instance, the balloon membrane 1106 includes proximal and distal sidewalls (e.g., edges of successive windings or passes of the membrane 1106) that are coupled together to accordingly reinforce the membrane 1106 and constrain inflation of the membrane 1106 into the specified balloon profile. The sidewalls of the balloon membrane 1106 extend helically with the balloon membrane 1106 and provide a helical structural support extending along the catheter shaft 1102. Inflation of the balloon assembly 1104 (including a single or multi-cellular balloon helically wrapped along the shaft) is constrained to the specified balloon profile of the component balloon membrane 1106 helically wrapped around the catheter shaft 1102.

Similar to the embodiments shown in FIGS. 9 and 10, the balloon assembly 1104 shown in FIG. 11 is optionally provided with a supplemental structural support, such as a jacket. The jacket optionally includes a coating applied along the balloon membrane 1106 to provide an isodiametric and smooth profile to the balloon membrane 1106. In still other examples, the jacket applied around the balloon membrane 1106 includes, but is not limited to, one or more of an interlaced jacket including a plurality of interlaced filaments as described herein.

In still other examples, any of the balloon assemblies described herein optionally have a jacket, including one or more of the jackets, interlaced jackets, coatings or the like used in combination with the remainder of the balloon assembly (e.g., one or more of a balloon membrane, foam substrate or the like).

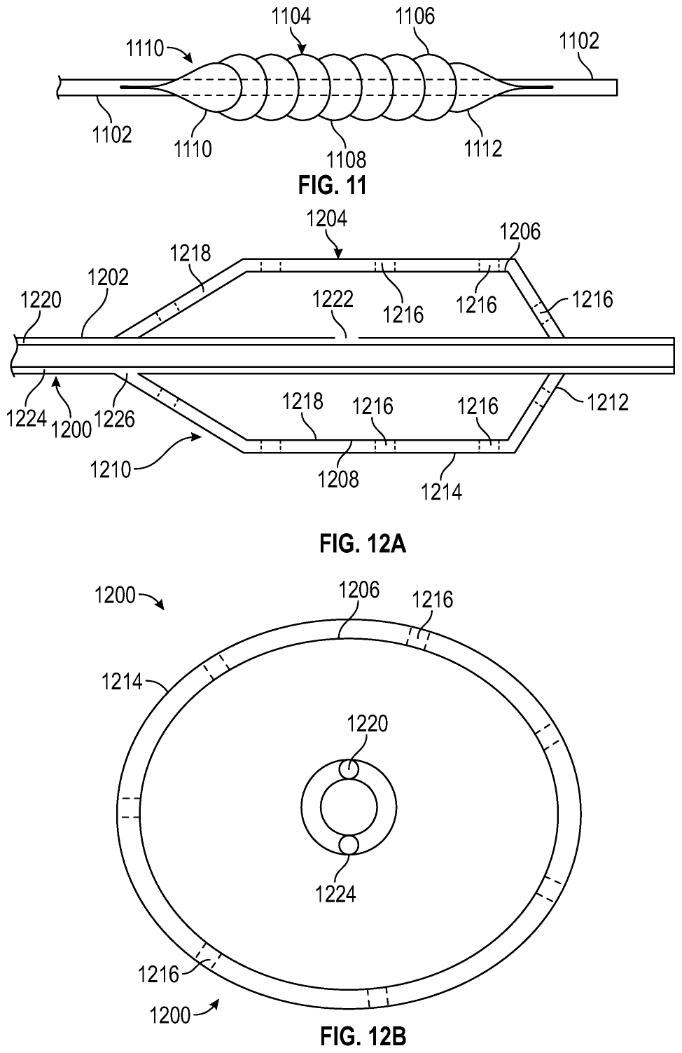

FIGS. 12A and 12B show a balloon catheter 1200 including a structural support 1214 coupled with a balloon such as the balloon membrane 1206. Referring first to FIG. 12A, the balloon catheter 1200 is shown in a first cross-sectional view extending along the longitudinal axis of the catheter shaft 1202. The balloon membrane 1206 of the balloon assembly 1204 is coupled along the catheter shaft 1202. For instance, the balloon proximal nose 1210 is coupled at one portion of the catheter shaft 1202 and the balloon distal nose 1212 is coupled with a distal portion of the catheter shaft 1202 relative to the balloon proximal nose 1210. The balloon body 1208 is interposed between the balloon proximal and distal noses 1210, 1212. The balloon membrane 1206 includes one or more of a compliant balloon membrane, semi-compliant balloon membrane or the like.

The balloon membrane 1206 including a compliant or semi-compliant membrane works in cooperation with the structural support 1214 to evenly distribute forces across the balloon membrane 1206 to the structural support 1214. The structural support 1214 is another example of a jacket and includes a supplemental balloon coupled along an exterior of the balloon membrane 1206. The structural support 1214 (e.g., a supplemental semi-compliant or non-compliant balloon) is coupled with the catheter shaft 1202, for instance proximally relative to the balloon proximal nose 1210 and distally relative to the balloon distal nose 1212. In examples, the balloon membrane 1206 and the structural support 1214 are each coupled with the catheter shaft 1202 with the same or differing features. For instance, one or more of adhesives, welds, crimps, clamps or the like are used to couple one or more of the balloon membrane 1206 and the structural support 1214 to the catheter shaft 1202.

Referring again to FIG. 12A, each of the balloon membrane 1206 and the structural support 1214 (the semi-compliant or non-compliant balloon) optionally includes dedicated inflation lumens 1220, 1224. In one example, the inflation lumens 1220, 1224 are provided in the catheter shaft (e.g., in a sidewall, lumens within a delivery lumen or the like). For instance, the balloon membrane 1206 is in communication with a first inflation lumen 1220 extending through the catheter shaft 1202. A first inflation port 1222 provides an opening from the first inflation lumen 1220 to the interior of the balloon membrane 1206. As further shown in FIG. 12A, a second inflation lumen 1224 extends through the catheter shaft 1202 to a second inflation port 1226. The second inflation port 1226 opens into the structural support 1214, for instance a space between the structural support 1214 (a balloon) and the balloon membrane 1206.

One or more bonding locations 1216 are optionally provided between the balloon membrane 1206 and the structural support 1214 (e.g., the support is another example of a jacket). The structural support 1214 and the balloon membrane 1206 include one or more decoupled spans 1218 extending between the bond locations 1216. The decoupled spans 1218 allow movement of the balloon membrane 1206 during inflation relative to the structural support 1214 to distribute pressures across portions of the structural support 1214 overlying the decoupled spans.

In other examples, the bonding locations for the structural support 1214 are localized to the catheter shaft 1202, for instance adjacent to the balloon proximal and distal noses 1210, 1212. In this configuration, the structural support 1214 and the balloon membrane 1206 are configured to move relative to each other along the length of each of the balloon membrane 1206 and the structural support 1214. For instance, during inflation of the balloon membrane 1206 and the structural support 1214 the balloon membrane moves relative to the structural support 1214 and pressure is evenly distributed throughout the membrane and absorbed by the structural support 1214.

In operation the balloon membrane 1206 and the balloon structural support 1214 are inflated and deflated together. A pressurized fluid is delivered through each of the first and second inflation points 1222, 1226 from the respective inflation lumens 1220, 1224. The balloon assembly 1204 including the balloon membrane 1206 and the structural support 1214 are inflated and deflated to and from the deployed configuration (FIG. 12A). In other examples, the inflation of the balloon membrane 1206 and the structural support 1214 are staggered with one of the membrane 1206 or the support inflated first and the other of the support 1214 or the membrane 1206 inflated second. In another example, inflation is alternated between the membrane 1206 and the support 1214.

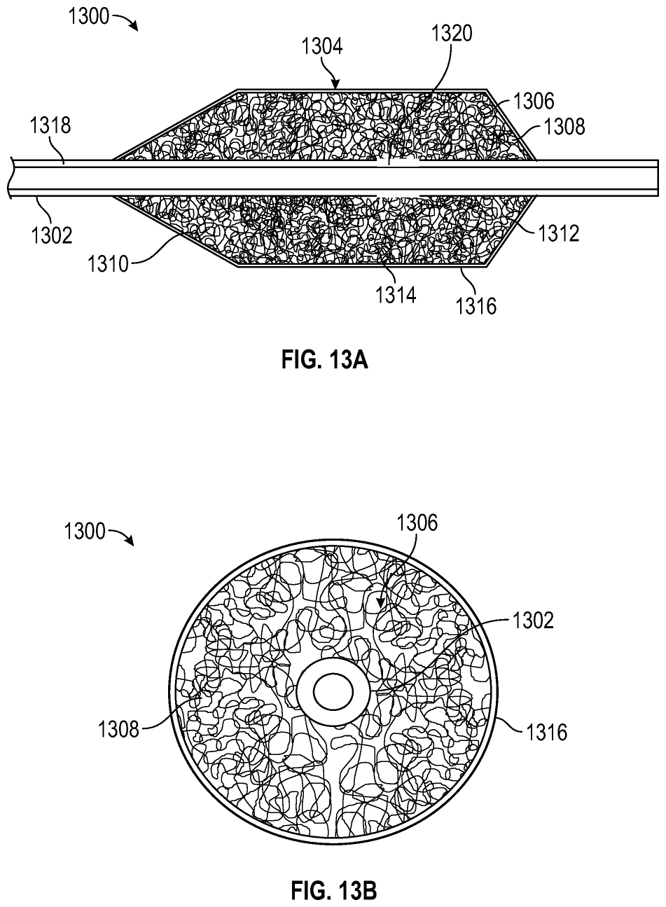

FIGS. 13A-C show another example of a balloon catheter 1300 having a balloon assembly 1304 including a foam substrate balloon. As shown in FIGS. 13A and 13B, the balloon assembly 1304 includes a foam substrate 1306 having a plurality of reticulations 1308. The reticulations 1308 are in communication with the inflation lumen 1318 of the catheter shaft 1302 through one or more inflation ports 1320 provided along the catheter shaft 1302. In the example shown in FIG. 13A, the inflation ports 1320 are localized to a middle portion of the catheter shaft 1302 within the assembly body 1314 between the assembly proximal and distal noses 1310, 1312. In other examples, a plurality of inflation ports 1320 are provided along the catheter shaft 1302 between the assembly proximal and distal noses 1310, 1312. Each of the ports 1320 is in communication directly or indirectly with one or more of the reticulations 1308 extending through the foam substrate 1306. The delivery of a pressurized fluid, for instance fluid delivered at pressures of 20 atmospheres or more (including 40 atmospheres, 60 atmospheres or more or the like), tills the reticulations 1308 and promotes inflation of the foam substrate 1306 into a specified balloon profile as shown in FIGS. 13A, B.

In another example, the foam substrate 1306 includes a foam elastomer having an unbiased configuration corresponding to the deployed configuration (and the specified balloon profile) shown in FIG. 13A, B. In this example, deflation of the foam substrate 1306 is accomplished with evacuation of the inflation fluid from the reticulations 1308. The evacuation of fluid biases the reticulations to compress and accordingly retracts the foam substrate 1306 into a compressed configuration shown in FIG. 13C.

The balloon assembly 1304 optionally includes one or more structural supports including, but not limited to, a jacket 1316, such as an interlaced jacket, coating, sleeve or the like. In one example, the jacket 1316 includes a coating or sleeve configured to close one or more of the reticulations 1308 along the exterior of the foam substrate 1306. The foam substrate 1306 is sealed to minimize leaks and the escape of one or more inflation fluids from the foam substrate 1306 during deployment or evacuation. Additionally, closing of the reticulations 1308 substantially prevents the drawing of fluids, such as body fluids, into the foam substrate 1306 during evacuation of inflation fluids from the foam substrate 1306. In still other examples, the structural support of the balloon assembly 1304 includes one or more of the interlaced jackets 1316 described herein.

Optionally, where deployment of the balloon assembly 1304 is desired from the compressed configuration shown in FIG. 13C, the evacuated inflation fluid is released and flows into the foam substrate 1306, for instance according to the elastic property of the foam substrate 1306. As the foam substrate 1306 and the reticulations therein expand, the reticulations 1308 draw in the inflation fluid and continue to open allowing the foam substrate 1306 to assume the deployed configuration shown in FIG. 13A including a specified balloon profile. In another example, the previously evacuated inflation fluid is delivered to the foam substrate 1306, for instance at a low pressure. The pressurized fluid provides a push to the elastomer of the foam substrate 1306 and prompts the expansion of the reticulations 1308 and corresponding expansion of the foam substrate 1306 into the deployed configuration show in FIGS. 13A and 13B.

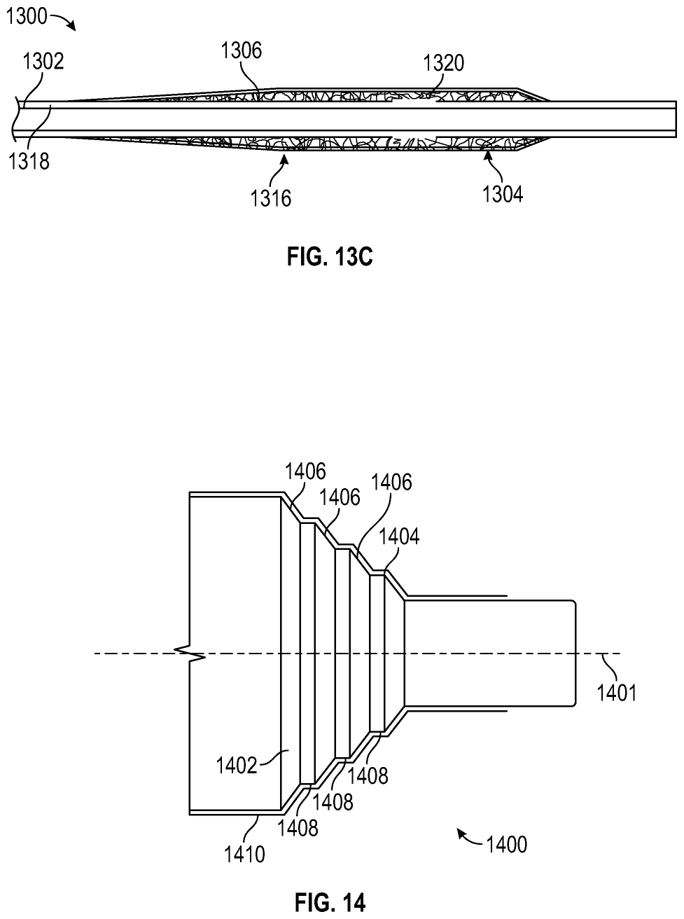

FIG. 14 shows a detailed view of another example of a balloon assembly 1400. As shown, the balloon assembly 1400 includes a balloon membrane shown in part by the balloon distal nose 1402. In this example, the balloon distal nose 1402 has a blunt profile, for instance, to facilitate use of the balloon assembly 1400 in one or more procedures such as a valve replacement procedure. The interlaced jacket 1410 includes one or more filaments in an interlacing pattern extending around the balloon membrane including the balloon distal nose 1402. In some examples, the coupling of the filaments along the balloon distal nose 1402 is frustrated by the steep taper of the blunt balloon distal nose 1402. The filaments gather, spread, fray, wrinkle, fold or the like because of the abrupt transition of diameters.

In one example, a terraced profile 1404 is provided along at least a portion of the balloon distal nose 1402. The terraced profile 1404 provides one or more anchor surfaces 1408 to facilitate the coupling of the interlaced jacket 1410 in place along the balloon distal nose 1402. The terraced profile 1404 further includes a plurality of tapered surfaces 1406 interspersed between the anchor surfaces 1408. As shown, the anchor surfaces 1408 are relatively horizontal in comparison to the tapered surfaces 1406. As described herein, the filaments of the interlaced jacket 1410 are coupled (e.g., bonded) along the anchor surfaces 1406 to fix the filaments in position and minimize spreading, gathering, fraying or the like.

The tapered surfaces 1406 are inclined relative to the anchor surfaces 1406. The taper of the tapered surfaces 1406 in combination with the anchor surfaces 1408 provides a composite angle for the balloon distal nose 1402 that is steep (e.g., 45 degrees) and at the same time bonds the filaments to the nose. For instance, the tapered surfaces 1406 are in one example at relatively steep component angles, such as 55 degrees relative to a catheter axis 1401. The steep tapered surfaces 1406 and the anchor surfaces 1408 (at a shallower angle including horizontal) provide an overall composite angle of the balloon distal nose 1402 of around 45 degrees. The anchor surfaces 1408 facilitate the coupling of the interlaced jacket 1410 including one or more filaments along the distal nose 1402 while the tapered surfaces provide a steep taper to offset the shallow anchor surfaces 1408. Accordingly, the balloon distal nose 1402 provides a relatively abrupt taper and diameter change while minimizing one or more bunching, spreading, fraying or the like of the interlaced jacket 1410. Accordingly, with the terraced profile 1404, in one example, blunt profiles are optionally provided along one or more of the noses of a balloon assembly 1400.

### Active Agents

In some embodiments, one or more of the layers herein (such as extruded layers, fibrous layers, polyurethane and/or epoxy layers, or the like) can include an active agent. The active agent can be configured to elute off the balloon after the balloon has been inserted into the patient vasculature.

It will be appreciated that active agents can include agents having many different types of activities. In some embodiments, active agents can include, but are not limited to, antiproliferatives such as paclitaxel, sirolimus (rapamycin), everolimus, biolimus A9, zotarolimus, tacrolimus, and pimecrolimus and mixtures thereof; analgesics and anti-inflammatory agents such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; anti-arrhythmic agents such as amiodarone HCl, disopyramide, flecainide acetate, quinidine sulphate; anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; anti-coagulants such as dicoumarol, dipyridamole, nicoumalone, phenindione; anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; anti-muscarinic agents: atropine, benzhexol HCl, biperiden, ethopropazine HCl, hyoscyamine, mepenzolate bromide, oxyphencylcimine HCl, tropicamide; anti-neoplastic agents and immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; beta-blockers such as acebutolol, alprenolol, atenolol, labetalol, metoprolol, nadolol, oxprenolol, pindolol, propranolol; cardiac inotropic agents such as amrinone, digitoxin, digoxin, enoximone, lanatoside C, medigoxin; corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; lipid regulating agents such as bezafibrate, clofibrate, fenofibrate, gemfibrozil, probucol; nitrates and other anti-anginal agents such as amyl nitrate, glyceryl trinitrate, isosorbide dinitrate, isosorbide mononitrate, pentaerythritol tetranitrate.

Other active agents include, but are not limited to, active agents for treatment of hypertension (HTN) such as guanethidine.

In a particular embodiment, the active agent includes paclitaxel, sirolimus (rapamycin), everolimus, biolimus A9, zotarolimus, tacrolimus, and pimecrolimus and mixtures thereof.

### Cross-Linking Materials

Various cross-linking materials can be used with embodiments herein. In some embodiments, cross-linking materials may be applied as a distinct layer between two other material layers. In other embodiments, cross-linking materials can be mixed or otherwise blended in with materials of other layers.

In some embodiments, cross-linking agents used in accordance with embodiments herein can include those with at least two photoreactive groups. Exemplary cross-linking agents are described in U.S. Publ. Pat. App. No. 2011/0245367, the content of which is herein incorporated by reference in its entirety.

In some embodiments, the first and/or second crosslinking agent can have a molecular weight of less than about 1500 kDa. In some embodiments, the crosslinking agent can have a molecular weight of less than about 1200, 1100, 1000, 900, 800, 700, 600, 500, or 400.

In some embodiments, at least one of the first and second cross-linking agents comprising a linking agent having formula. Photo¹-LG-Photo², wherein Photo¹ and Photo², independently represent at least one photoreactive group and LG represents a linking group comprising at least one silicon or at least one phosphorus atom, there is a covalent linkage between at least one photoreactive group and the linking group, wherein the covalent linkage between at least one photoreactive group and the linking group is interrupted by at least one heteroatom.

In some embodiments, at least one of the first and second cross-linking agents comprising a linking agent having a formula selected from:

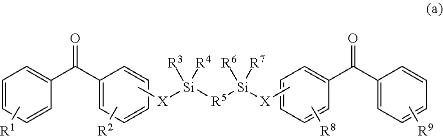

wherein R1, R2, R8 and R9 are H, methyl, ethyl, or any substitution; R3, R4, R6 and R7 are alkyl, aryl, or a combination thereof; R5 is C1-C20 alkyl or aryl or any substitution; and each X, independently, is O, N Se, S, or alkyl, or a combination thereof;

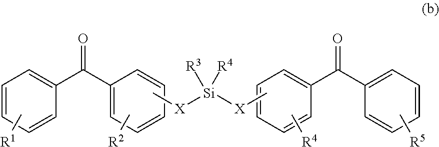

wherein R1, R2, R4, R5 are H, methyl, ethyl, or any substitution; R3 can be alkyl, aryl, or a combination thereof; and X, independently, are O, N, Se, S, alkylene, or a combination thereof;

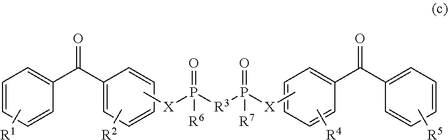

wherein R1, R2, R4 and R5 are H, methyl, ethyl, or any substitution; R3 is C1-C20 alkyl or aryl or any substitution; R6 and R7 are alkyl, aryl, or a combination thereof; and each X can independently be O, N. Se, S, alkylene, or a combination thereof; and

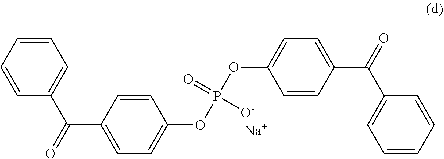 [SMILES: O=C(c1ccccc1)c1ccc(OP(=O)([O-])Oc2ccc(C(=O)c3ccccc3)cc2)cc1 ; [Na+]]

In a particular embodiment, the cross-linking agent can be bis(4-benzoylphenyl) phosphate.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁—Y—X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzolbenzyldimethylammonio)ethyl]-4-benzoylbenzlmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

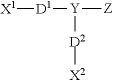

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can he the same or different.

In other embodiments, the non-ionic photoactivatable cross-linking agent can be represented by the formula:

PG²−LE²−X−LE¹−PG¹

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹−X−R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis(4-benzoylbenzyl ether) of pentaerythritol (tetrakis(4-benzoylphenylmethoxymethyl)methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis (4-benzoylbenzyldimethylammonium) dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in U.S. 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

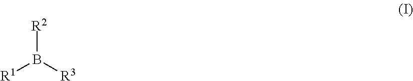

wherein R¹ is a radical comprising a photoreactive group; R² is selected from OH and a radical comprising a photoreactive group, an akyl group and an aryl group; and R³ is selected from OH and a radical comprising a photoreactive group. In some embodiments the bonds B—R¹, B—R² and B—R³ can be chosen independently to be interrupted by a heteroatom, such as O, N, S, or mixtures thereof.

Additional agents for use with embodiments herein can include stilbene-based reactive compounds including, but not limited to, those disclosed in U.S. 61/736,436, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, cross-linking agents, and associated reagents are disclosed in US2011/0059874; US 2011/0046255; and US 2010/0198168, the content of all of which is herein incorporated by reference.

### Photoreactive Groups

Various components used in compositions herein can include photoreactive groups. As used herein, the phrases “latent photoreactive group” and “photoreactive group” are used interchangeably and refer to a chemical moiety that is sufficiently stable to remain in an inactive state (i.e., ground state) under normal storage conditions but that can undergo a transformation from the inactive state to an activated state when subjected to an appropriate energy source. Unless otherwise stated, references to photoreactive groups herein shall also include the reaction products of the photoreactive groups. Photoreactive groups respond to specific applied external stimuli to undergo active specie generation with resultant covalent bonding to an adjacent chemical structure. For example, a photoreactive group can be activated and can abstract a hydrogen atom from an alkyl group. A covalent bond can then form between the compound with the photoreactive group and the compound with the C—H bond. Suitable photoreactive groups are described in U.S. Pat. No. 5,002,582, the disclosure of which is incorporated herein by reference.

Photoreactive groups can be chosen to be responsive to various portions of actinic radiation. For example, groups can be chosen that can be photoactivated using either ultraviolet or visible radiation. Suitable photoreactive groups include, for example, azides, diazos, diazirines, ketones, and quinones. The photoreactive groups generate active species such as free radicals including, for example, nitrenes, carbenes, and excited states of ketones upon absorption of electromagnetic energy.

The photoreactive group can comprise an aryl ketone, such as acetophenone, benzophenone, anthrone, and anthrone-like heterocycles (i.e., heterocyclic analogs of anthrone such as those having N, O, or S in the 10-position), or their substituted (e.g., ring substituted) derivatives. Examples of aryl ketones include heterocyclic derivatives of anthrone, including acridone, xanthone, and thioxanthone, and their ring substituted derivatives. Other suitable photoreactive groups include quinone such as, for example anthraquinone.

The functional groups of such aryl ketones can undergo multiple activation/inactivation/reactivation cycles. For example, benzophenone is capable of photochemical excitation with the initial formation of an excited singlet state that undergoes intersystem crossing to the triplet state. The excited triplet state can insert into carbon-hydrogen bonds by abstraction of a hydrogen atom (from a polymeric coating layer, for example), thus creating a radical pair. Subsequent collapse of the radical pair leads to formation of a new carbon-carbon bond. If a reactive bond (e.g., carbon/hydrogen) is not available for bonding, the ultraviolet light-induced excitation of the benzophenone group is reversible and the molecule returns to ground state energy level upon removal of the energy source. Photoreactive aryl ketones such as benzophenone and acetophenone can undergo multiple reactivations in water and hence can provide increased coating efficiency.

The azides constitute another class of photoreactive groups and include arylazides (C₆R₅N₃) such as phenyl azide and 4-fluoro-3-nitrophenyl azide; acyl azides (—CO—N₃) such as benzoyl azide and p-methylbenzoyl azide; azido formates (—O—CO—N₃) such as ethyl azidoformate and phenyl azidoformate; sulfonyl azides (—SO₂—N₃) such as benzenesulfonyl azide; and phosphoryl azides (RO)₂PON₃ such as diphenyl phosphoryl azide and diethyl phosphoryl azide.

Diazo compounds constitute another class of photoreactive groups and include diazoalkanes (—CHN₂) such as diazomethane and diphenyldiazornethane; diazoketones (—CO—CHN₂) such as diazoacetophenone and 1-trifluoromethyl-1-diazo-2-pentanone; diazoacetates (—O—CO—CHN₂) such as t-butyl diazoacetate and phenyl diazoacetate; and beta-keto-alpha-diazoacetates (—CO—CN₂—CO—O—) such as t-butyl alpha diazoacetoacetate.

Other photoreactive groups include the diazirines (—CHN₂) such as 3-trifluoromethyl-3-phenyldiazirine; and ketenes (—CH═C═O) such as ketene and diphenylketene.

In some aspects, the photoreactive groups can be aryl ketones, such as benzophenone.

### VARIOUS NOTES & EXAMPLES

Example 1 can include subject matter such as a balloon catheter comprising: a catheter shaft extending between proximal and distal end portions along a shaft axis, the catheter shaft includes: an inflation lumen, and at least one inflation port in communication with the inflation lumen; and a balloon assembly coupled with the catheter shaft and in communication with the at least one inflation port, the balloon assembly includes: a balloon membrane having a balloon body, a balloon proximal nose and a balloon distal nose coupled with the catheter shaft, and an interlaced jacket coupled with the balloon membrane, the interlaced jacket includes interlaced filaments extending at diverging angles relative to the shaft axis.

Example 2 can include, or can optionally be combined with the subject matter of Example 1, to optionally include wherein the interlaced jacket is bonded with the balloon membrane.

Example 3 can include, or can optionally be combined with the subject matter of one or any combination of Examples 1 or 2 to optionally include wherein the interlaced jacket is bonded with the balloon membrane with one or more of a dispersion infiltrated through the interlaced jacket to the balloon membrane or an adhesive between the balloon membrane and the interlaced jacket.

Example 4 can include, or can optionally be combined with the subject matter of one or any combination of Examples 1-3 to optionally include wherein the interlaced jacket is continuously bonded along the balloon membrane.

Example 5 can include, or can optionally be combined with the subject matter of one or any combination of Examples 1-4 to optionally include wherein the interlaced jacket is bonded at one or more perimeter locations around a perimeter of the balloon membrane.

Example 6 can include, or can optionally be combined with the subject matter of Examples 1-5 to optionally include wherein the balloon membrane is an elastic membrane, and the balloon membrane is configured to move relative to the interlaced jacket between the one or more perimeter locations.

Example 7 can include, or can optionally be combined with the subject matter of Examples 1-6 to optionally include wherein the interlaced jacket is coupled along an exterior of the balloon membrane.

Example 8 can include, or can optionally be combined with the subject matter of Examples 1-7 to optionally include wherein the interlaced jacket extends continuously around the balloon membrane.

Example 9 can include, or can optionally be combined with the subject matter of Examples 1-8 to optionally include wherein the interlaced filaments extending at diverging angles relative to the catheter shaft are misaligned with the catheter shaft.

Example 10 can include, or can optionally be combined with the subject matter of Examples 1-9 to optionally include wherein the diverging angles of the interlaced filaments include diverging angles of around 40 to 70 degrees.

Example 11 can include, or can optionally be combined with the subject matter of Examples 1-10 to optionally include wherein the diverging angles of the interlaced filaments include diverging angles of around 50 to 60 degrees.

Example 12 can include, or can optionally be combined with the subject matter of Examples 1-11 to optionally include wherein the interlaced filaments are in one or more interlacing configurations including braiding, weaving, knitting, crocheting, nalbinding, mesh or non-woven.

Example 13 can include, or can optionally be combined with the subject matter of Examples 1-12 to optionally include wherein the interlaced jacket is coupled with the balloon membrane at the balloon body.

Example 14 can include, or can optionally be combined with the subject matter of Examples 1-13 to optionally include wherein the interlaced jacket is coupled with the balloon membrane at one or more of the balloon body, the balloon distal nose or the balloon proximal nose.

Example 15 can include, or can optionally be combined with the subject matter of Examples 1-14 to optionally include wherein at least one of the balloon distal nose and the balloon proximal nose include a terraced profile having tapered surfaces with interposing anchor surfaces.

Example 16 can include, or can optionally be combined with the subject matter of Examples 1-15 to optionally include wherein the interlaced jacket conforms to the ten⁻aced profile and the interlaced jacket is anchored along the interposing anchor surfaces.

Example 17 can include, or can optionally be combined with the subject matter of Examples 1-16 to optionally include wherein balloon distal nose includes a blunt profile and the balloon proximal nose includes a tapered profile relative to the blunt profile.

Example 18 can include, or can optionally be combined with the subject matter of Examples 1-17 to optionally include wherein the interlaced jacket conforms to at least the blunt profile of the of the balloon distal nose.

Example 19 can include, or can optionally be combined with the subject matter of Examples 1-18 to optionally include wherein the interlaced jacket includes at least first and second filament densities of the interlaced filaments, the first filament density is different than the second filament density.

Example 20 can include, or can optionally be combined with the subject matter of Examples 1-19 to optionally include wherein the first filament density is a first pic count and the second filament density is a second pic count.

Example 21 can include, or can optionally be combined with the subject matter of Examples 1-20 to optionally include wherein the balloon membrane proximate at least one of the balloon distal and balloon proximal noses includes the first filament density and the balloon membrane proximate the balloon body includes the second filament density.

Example 22 can include, or can optionally be combined with the subject matter of Examples 1-21 to optionally include wherein the first filament density is greater than the second filament density.

Example 23 can include, or can optionally be combined with the subject matter of Examples 1-22 to optionally include wherein balloon assembly is configured for inflation to at least 20 atmospheres.

Example 24 can include, or can optionally be combined with the subject matter of Examples 1-23 to optionally include wherein the balloon membrane includes a semi-compliant balloon membrane, a non-compliant balloon membrane or a compliant balloon membrane.

Example 25 can include, or can optionally be combined with the subject matter of Examples 1-24 to optionally include a balloon catheter comprising: a catheter shaft extending between proximal and distal end portions along a shaft axis, the catheter shaft includes: an inflation lumen, and at least one inflation port in communication with the inflation lumen; and a balloon assembly coupled with the catheter shaft, the balloon assembly includes: a balloon membrane in communication with the at least one inflation port, the balloon membrane includes a balloon body, a balloon proximal nose and a balloon distal nose coupled with the catheter shaft, and at least one structural support coupled with the balloon membrane.

Example 26 can include, or can optionally be combined with the subject matter of Examples 1-25 to optionally include wherein the balloon membrane includes a compliant balloon membrane, and the at least one structural support includes one or more of a semi-compliant or non-compliant balloon coupled with the compliant balloon membrane, the semi-compliant balloon constrains inflation of the compliant balloon membrane to a specified balloon profile.

Example 27 can include, or can optionally be combined with the subject matter of Examples 1-26 to optionally include wherein the semi-compliant balloon is continuously coupled along the compliant balloon membrane.

Example 28 can include, or can optionally be combined with the subject matter of Examples 1-27 to optionally include wherein the semi-compliant balloon is coupled along the compliant balloon membrane at a plurality of bond locations with decoupled spans therebetween.

Example 29 can include, or can optionally be combined with the subject matter of Examples 1-28 to optionally include wherein the balloon assembly includes a balloon proximal nose and a balloon distal nose, and the semi-compliant balloon is coupled with the compliant balloon membrane proximate to the balloon proximal nose and the balloon distal nose of the balloon assembly with a decoupled span therebetween.

Example 30 can include, or can optionally be combined with the subject matter of Examples 1-29 to optionally include wherein the balloon assembly includes a balloon proximal nose and a balloon distal nose, and the compliant balloon membrane and the semi-compliant balloon membrane include one or more decoupled spans between the balloon proximal nose and the balloon distal nose, and the compliant balloon membrane is configured to move relative to the semi-compliant balloon membrane across the decoupled spans.

Example 31 can include, or can optionally be combined with the subject matter of Examples 1-30 to optionally include wherein the catheter shaft includes a second inflation lumen and a second inflation port in communication with the second inflation lumen, and the second inflation port is in communication with the semi-compliant balloon membrane, the inflation port is in communication with the compliant balloon membrane; and wherein an interior of the semi-compliant balloon membrane is isolated from an interior of the compliant balloon membrane.

Example 32 can include, or can optionally be combined with the subject matter of Examples 1-31 to optionally include wherein the at least one structural support includes one or more struts extending from the catheter shaft to the balloon membrane.

Example 33 can include, or can optionally be combined with the subject matter of Examples 1-32 to optionally include wherein the balloon membrane includes: a first balloon cell extending along the catheter shaft, a second balloon cell extending along the catheter shaft, and wherein the one or more struts include side walls of the first and second balloon cells extending from the catheter shaft to a perimeter of the balloon membrane.

Example 34 can include, or can optionally be combined with the subject matter of Examples 1-33 to optionally include wherein at least one of the side walls of the first and second balloon cells include a common side wall shared by the first and second balloon cells.

Example 35 can include, or can optionally be combined with the subject matter of Examples 1-34 to optionally include wherein the first and second balloon cells are coextruded first and second balloon cells.

Example 36 can include, or can optionally be combined with the subject matter of Examples 1-35 to optionally include wherein the at least one structural support includes a jacket coupled around the first and second balloon cells, the jacket configured to constrain inflation of the first and second balloon cells to a specified balloon profile.

Example 37 can include, or can optionally be combined with the subject matter of Examples 1-36 to optionally include wherein the jacket includes an interlaced jacket having interlaced filaments extending at diverging angles relative to the catheter shaft.

Example 38 can include, or can optionally be combined with the subject matter of Examples 1-37 to optionally include wherein the balloon membrane includes at least one balloon cell helically wound along the catheter shaft, and the one or more struts includes a side wall of the at least one balloon cell.

Example 39 can include, or can optionally be combined with the subject matter of Examples 1-38 to optionally include wherein the side wall of the helically wound at least one balloon cell is bonded between successive windings of the at least one balloon cell.

Example 40 can include, or can optionally be combined with the subject matter of Examples 1-39 to optionally include wherein an exterior of the helically wound at least one balloon cell relative to the catheter shaft is the specified balloon profile of the balloon membrane.

Example 41 can include, or can optionally be combined with the subject matter of Examples 1-40 to optionally include wherein the at least one structural support includes a jacket coupled around an exterior of the helically wound at least one balloon cell relative to the catheter shall, and the jacket is configured to constrain inflation of the at least one balloon cell to a specified balloon profile.

Example 42 can include, or can optionally be combined with the subject matter of Examples 1-41 to optionally include wherein the jacket includes a coating.

Example 43 can include, or can optionally be combined with the subject matter of Examples 1-42. to optionally include wherein the jacket includes an interlaced jacket having interlaced filaments extending at diverging angles relative to the catheter shaft.

Example 44 can include, or can optionally be combined with the subject matter of Examples 1-43 to optionally include a balloon catheter comprising: a catheter shaft extending between proximal and distal end portions along a shaft axis, the catheter shaft includes: a lumen configured for conveying fluid, and at least one fluid port in communication with the lumen; and a balloon assembly coupled with the distal end portion and in communication with the at least one fluid port, the balloon assembly includes: a compressible substrate coupled with the catheter shaft, the compressible substrate includes a substrate body, a substrate proximal nose and a substrate distal nose, and reticulated pores in the compressible substrate, the reticulated pores in communication with the at least one fluid port.

Example 45 can include, or can optionally be combined with the subject matter of Examples 1-44 to optionally include wherein the compressible substrate includes a foam elastomer.

Example 46 can include, or can optionally be combined with the subject matter of Examples 1-45 to optionally include wherein the balloon assembly includes deployed and compressed configurations: in the deployed configuration the reticulated pores are open and the compressible substrate is in a specified balloon profile larger than a shaft profile of the catheter shaft, and in the compressed configuration the reticulated pores are closed and the compressible substrate is in a compressed profile smaller than the specified balloon profile and proximate the shaft profile.

Example 47 can include, or can optionally be combined with the subject matter of Examples 1-46 to optionally include wherein in the deployed configuration the open reticulated pores are filled with a fluid, and in the compressed configuration the fluid is drawn from the closed reticulated pores.

Example 48 can include, or can optionally be combined with the subject matter of Examples 1-47 to optionally include a vacuum source coupled with the lumen of the catheter shaft, the vacuum source is configured to transition the balloon assembly between the deployed and compressed configurations by drawing fluid from the reticulated pores.

Example 49 can include, or can optionally be combined with the subject matter of Examples 1-48 to optionally include wherein the compressible substrate includes an elastomer and the compressible substrate is configured to transition from the compressed configuration toward the deployed configuration according to the elastomer.

Example 50 can include, or can optionally be combined with the subject matter of Examples 1-49 to optionally include a fluid source coupled with the lumen of the catheter shaft, the fluid source is configured to transition the balloon assembly between the compressed and deployed configurations by delivering fluid to the reticulated pores.

Example 51 can include, or can optionally be combined with the subject matter of Examples 1-50 to optionally include wherein the balloon assembly includes a jacket around the compressible substrate.

Example 52 can include, or can optionally be combined with the subject matter of Examples 1-51 to optionally include wherein the jacket includes an interlaced jacket having interlaced filaments extending at diverging angles relative to the catheter shaft.

Example 53 can include, or can optionally be combined with the subject matter of Examples 1-52 to optionally include wherein the jacket closes the reticulated pores along a perimeter surface of the compressible substrate.

Example 54 can include, or can optionally be combined with the subject matter of Examples 1-53 to optionally include wherein the substrate distal nose includes a blunt profile and the substrate proximal nose includes a tapered profile relative to the blunt profile, and the compressible substrate is shaped to include each of the blunt and tapered profiles.

Example 55 can include, or can optionally be combined with the subject matter of Examples 1-54 to optionally include wherein at least one of the substrate distal nose and the substrate proximal nose includes a terraced profile having tapered surfaces with interposing anchor surfaces, and the compressible substrate is shaped to include the terraced profile.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a ten in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R.§ 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

What is claimed is:

1. A balloon catheter comprising:
a catheter shaft extending between proximal and distal end portions along a shaft axis, the catheter shaft includes:
an inflation lumen, and
at least one inflation port n communication with the inflation lumen; and
a balloon assembly coupled with the catheter shaft and in communication with the at least one inflation port, the balloon assembly includes:
a balloon membrane having a balloon body, a balloon proximal nose and a balloon distal nose coupled with the catheter shaft, and
an interlaced jacket coupled with the balloon membrane, the interlaced jacket includes interlaced filaments extending at diverging angles relative to the shaft axis.

2. The balloon catheter of claim 1, herein the interlaced jacket is bonded with the balloon membrane.

3. The balloon catheter of claim 2, wherein the interlaced jacket is bonded with the balloon membrane with one or more of a dispersion infiltrated through the interlaced jacket to the balloon membrane or an adhesive between the balloon membrane and the interlaced jacket.

4. The balloon catheter of claim 1, wherein the interlaced jacket is continuously bonded along the balloon membrane.

5. The balloon catheter of claim 1, wherein the interlaced jacket is bonded at one or more perimeter locations around a perimeter of the balloon membrane.

6. The balloon catheter of claim 5, wherein the balloon membrane is an elastic membrane, and the balloon membrane is configured to move relative to the interlaced jacket between the one or more perimeter locations.

7. The balloon catheter of claim 1, wherein the interlaced jacket is coupled along an exterior of the balloon membrane.

8. The balloon catheter of claim 1, wherein the interlaced jacket extends continuously around the balloon membrane.

9. The balloon catheter of claim 1, wherein the interlaced filaments extending at diverging angles relative to the catheter shaft are misaligned with the catheter shaft.

10. The balloon catheter of claim 1, wherein the diverging angles of the interlaced filaments include diverging angles of around 40 to 70 degrees.

11. The balloon catheter of claim 1, wherein the diverging angles of the interlaced filaments include diverging angles of around 50 to 60 degrees.

12. The balloon catheter of claim 1, wherein the interlaced filaments are in one or more interlacing configurations including braiding, weaving, knitting, crocheting, nalbinding, mesh or non-woven.

13. The balloon catheter of claim 1, wherein the interlaced jacket is coupled with the balloon membrane at the balloon body.

14. The balloon catheter of claim 1, wherein the interlaced jacket is coupled with the balloon membrane at one or more of the balloon body, the balloon distal nose or the balloon proximal nose.

15. The balloon catheter of claim 1, wherein at least one of the balloon distal nose and the balloon proximal nose include a terraced profile having tapered surfaces with interposing anchor surfaces.

16. The balloon catheter of claim 15, wherein the interlaced jacket conforms to the terraced profile and the interlaced jacket is anchored along the interposing anchor surfaces.

17. The balloon catheter of claim 1, wherein balloon distal nose includes a blunt profile and the balloon proximal nose includes a tapered profile relative to the blunt profile.

18. The balloon catheter of claim 17, wherein the interlaced jacket conforms to at least the blunt profile of the of the balloon distal nose.

19. The balloon catheter of claim 1, wherein the interlaced jacket includes at least first and second filament densities of the interlaced filaments, the first filament density is different than the second filament density.

20. The balloon catheter of claim 19, wherein the first filament density is a first pic count and the second filament density is a second pic count.

21. The balloon catheter of claim 19, wherein the balloon membrane proximate at least one of the balloon distal and balloon proximal noses includes the first filament density and the balloon membrane proximate the balloon body includes the second filament density.

22. The balloon catheter of claim 21, wherein the first filament density is greater than the second filament density.

23. The balloon catheter of claim 1, wherein balloon assembly is configured for inflation to at least 20 atmospheres.

24. The balloon catheter of claim 1, wherein the balloon membrane includes a semi-compliant balloon membrane, a non-compliant balloon membrane or a compliant balloon membrane.
